(19) United States
(12) Patent Application Publication
(10) Pub. No.: US 2008/0020987 A1
(43) Pub. Date: Jan. 24, 2008

(54) Processes for preparing pyrazole-O-glycoside derivatives and novel intermediates of said processes

(76) Inventor: Waldemar Pfrengle, Biberach (DE)

Correspondence Address: MICHAEL P. MORRIS;BOEHRINGER INGELHEIM CORPORATION, 900 RIDGEBURY ROAD, P. O. BOX 368, RIDGEFIELD, CT, 06877-0368, US

(21) Appl. No.: 11/490,426

(22) Filed: Jul. 20, 2006

(51) Int. Cl. A61K 31/7056; C07H 17/02

(52) U.S. Cl. 514/25; 536/17.4

(57) ABSTRACT

The present invention relates to processes for preparing the compounds of general formula I,

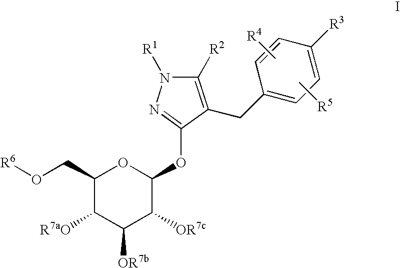

wherein the groups R¹ to R⁶ and R^{7a}, R^{7b}, R^{7c} are defined according to claim 1. Furthermore the present invention relates to processes for preparing educts and intermediates in the above processes and to intermediates as such.

This application claims priority benefit to EP 05 015 935, filed Jul. 22, 2005 and the contents of which are incorporated herein.

The present invention relates to processes for preparing of pyrazole-O-glycoside derivatives of the general formula (I),

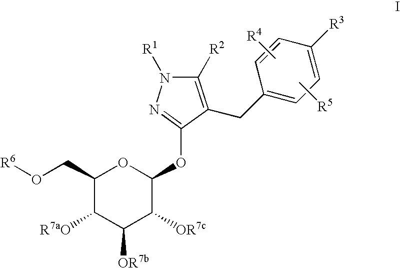

wherein the substituents R¹ to R⁶ and R^{7a}, R^{7b}, R^{7c} are defined as hereinafter.

Furthermore the present invention relates to processes for preparing compounds of the formula (III)

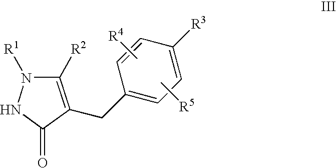

wherein R¹ to R⁵ are defined as hereinafter.

Furthermore the present invention relates to a process for preparing compounds of the formula (IV)

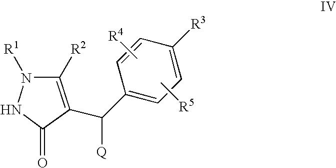

wherein R¹ to R⁵ and Q are defined as hereinafter.

Furthermore the present invention relates to a process for preparing a pyrazole derivative of the formula (XI)

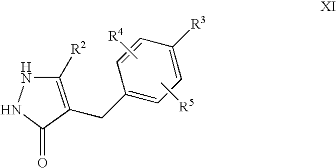

wherein R² to R⁵are defined as hereinafter.

Furthermore the present invention relates to novel intermediates and starting materials useful in the processes according to this invention.

### AIM OF THE INVENTION

The aim of the present invention is to find new processes for preparing of pyrazole-O-glycoside derivatives of the formula (I); in particular processes with which the product may be obtained in high yields and/or high chemical and diastereomeric purity and which allow the manufacture of the product in a commercial scale with a low technical expenditure and a high space/time yield.

Another aim of the present invention is to provide processes for preparing the starting materials of the beforementioned processes.

Further aims of the present invention relate to new intermediates and starting materials in the process according to the present invention.

Other aims of the present invention will become apparent to the skilled man directly from the foregoing and following description.

### OBJECT OF THE INVENTION

In a first aspect the present invention relates to a process for preparing the compounds of general formula (I),

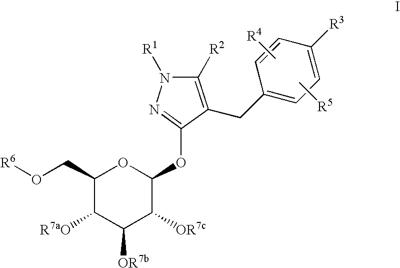

wherein

R¹ denotes C₁₋₄-alkyl, a C₁₋₄-alkyl group substituted with one or more fluorine atoms, or C₃₋₆-cycloalkyl; and

R² denotes C₁₋₄-alkyl, a C₁₋₄-alkyl group substituted with one or more fluorine atoms, or C₃₋₆-cycloalkyl; and

R³ denotes fluorine, chlorine, bromine, C₁₋₄-alkyl, C₃₋₆-cycloalkyl, C₁₋₄-alkoxy, or C₃₋₆-cycloalkyl-oxy; and

R⁴, R⁵ independently of one another denote hydrogen, fluorine, chlorine, bromine, C₁₋₄-alkyl, or C₁₋₄-alkoxy; and

R⁶, R^{7a}

R^{7b}, R^{7c} independently of one another have a meaning selected from the group hydrogen, (C₁₋₆-alkyl)carbonyl, phenylcarbonyl and phenyl-(C₁₋₃-alkyl)-carbonyl; including the tautomers, stereoisomers, mixtures thereof and the salts thereof;

characterised in that the aglycone of the formula (III)

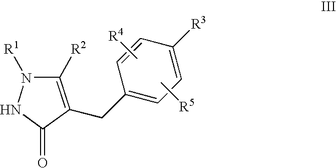

wherein R¹ to R⁵ are defined as hereinbefore;

is obtained by a catalytic hydrogenation of a compound of the formula (IV)

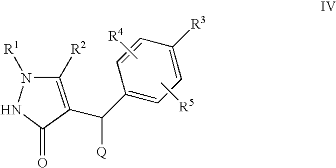

wherein R¹ to R⁵ are defined as hereinbefore, and

Q is Cl, Br, I, C₁₋₄-alkoxy, C₁₋₄-alkylthio, phenylthio, C₃₋₆-cycloalkyl-oxy, C₁₋₄-alkylcarbonyloxy, —NR^{a}R^{b}, wherein R^{a}, R^{b} independently of one another denote C₁₋₄-alkyl, or —NR^{a}R^{b} denotes pyrrolidinyl, piperidinyl, morpholinyl, piperazinyl or N—C₁₋₄-alkyl-piperazinyl;

in a solvent or mixture of solvents.

In a second aspect the present invention relates to a process for preparing compounds of the above given general formula (I), wherein R¹ to R⁵, R⁶, R^{7a}, R^{7b}, R^{7c} are defined as hereinbefore, including the tautomers, stereoisomers, mixtures thereof and the salts thereof;

characterized in that an aglycone of the formula (III)

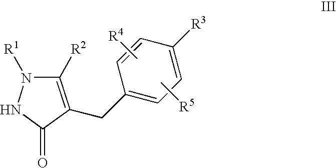

wherein R¹ to R⁵ are defined as hereinbefore;

is reacted with a glucose derivative of the formula (II)

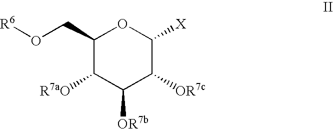

wherein

X denotes bromine or chlorine;

R⁶, R^{7a}, R^{7b} and R^{7c} independently of one another have a meaning selected from the group (C₁₋₆-alkyl)carbonyl, phenylcarbonyl and phenyl-(C₁₋₃-alkyl)-carbonyl;

in a solvent or mixture of solvents; and optionally the product of the formula (I), wherein substituents R⁶, R^{7a}, R^{7b}, R^{7c} are not hydrogen, is deprotected, particularly preferred by the process according to the third aspect of this invention.

In a third aspect the present invention relates to a process for preparing compounds of the formula (IH)

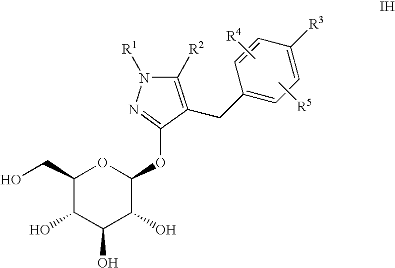

wherein R¹ to R⁵ are defined as hereinbefore and hereinafter,

including the tautomers, stereoisomers, mixtures thereof and the salts thereof;

comprising the step of deprotecting the compound of the formula (I)

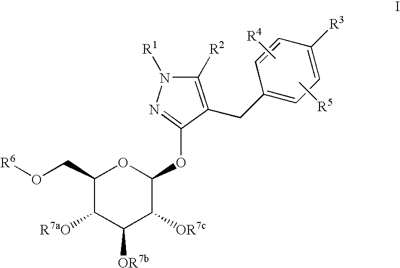

wherein R¹ to R⁵ are defined as hereinbefore and R⁶, R^{7a}, R^{7b} and R^{7c} are defined as hereinbefore and hereinafter but one or more of them not being hydrogen, by cleaving the substituents R⁶, R^{7a}, R^{7b} and R^{7c} not being hydrogen in a solvent or a mixture of solvents.

In a fourth aspect the present invention relates to a process for preparing compounds of the formula (III)

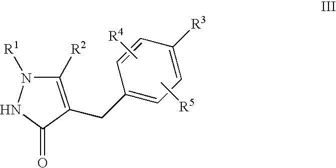

wherein

R¹ denotes C₁₋₄-alkyl, a C₁₋₄-alkyl group substituted with 1 to 3 fluorine atoms, or C₃₋₆-cycloalkyl; and

R² denotes C₁₋₄-alkyl, a C₁₋₄-alkyl group substituted with one or more fluorine atoms, or C₃₋₆-cycloalkyl; and

R³ denotes fluorine, chlorine, bromine, C₁₋₄-alkyl, C₃₋₆-cycloalkyl, C₁₋₄-alkoxy, or C₃₋₆-cycloalkyl-oxy; and

R⁴, R⁵ independently of one another denote hydrogen, fluorine, chlorine, bromine, C₁₋₄-alkyl, or C₁₋₄-alkoxy;

including the tautomers, stereoisomers, mixtures thereof and the salts thereof;

comprising the step of catalytically hydrogenating a pyrazole derivative of the formula (IV)

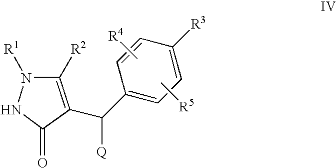

wherein R¹ to R⁵ are defined as hereinbefore; and

Q is Cl, Br, I, C₁₋₄-alkoxy, C₁₋₄-alkylthio, phenylthio, C₃₋₆-cycloalkyl-oxy, C₁₋₄-alkylcarbonyloxy, —NR^{a}R^{b}, wherein R^{a}, R^{b} independently of one another denote C₁₋₄-alkyl, or —NR^{a}R^{b} denotes pyrrolidinyl, piperidinyl, morpholinyl, piperazinyl or N—C₁₋₄-alkyl-piperazinyl,

in a solvent or a mixture of solvents.

In a fifth aspect the present invention relates to a process for preparing compounds of the formula (IV)

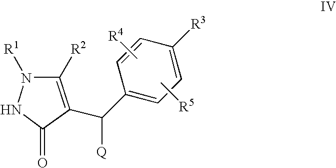

wherein

R¹ denotes C₁₋₄-alkyl, a C₁₋₄-alkyl group substituted with one or more fluorine atoms, or C₃₋₆-cycloalkyl; and

R² denotes C₁₋₄-alkyl, a C₁₋₄-alkyl group substituted with one or more fluorine atoms, or C₃₋₆-cycloalkyl; and

R³ denotes fluorine, chlorine, bromine, C₁₋₄-alkyl, C₃₋₆-cycloalkyl, C₁₋₄-alkoxy, or C₃₋₆-cycloalkyl-oxy; and

R⁴, R⁵ independently of one another denote hydrogen, fluorine, chlorine, bromine, C₁₋₄-alkyl, or C₁₋₄-alkoxy; and

Q is C₁₋₄-alkoxy, C₁₋₄-alkylthio, C₃₋₆-cycloalkyl-oxy, phenylthio, —NR^{a}R^{b}, wherein R^{a}, R^{b} independently of one another denote C₁₋₄-alkyl, or —NR^{a}R^{b} denotes pyrrolidinyl, piperidinyl, morpholinyl, piperazinyl or N—C₁₋₄-alkyl-piperazinyl;

including the tautomers, stereoisomers, mixtures thereof and the salts thereof;

comprising the step of reacting a pyrazole derivative of the formula (VI)

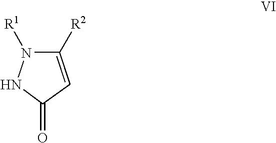

wherein R¹ and R² are defined as hereinbefore;

with a benzaldehyde derivative of the formula (V)

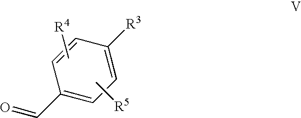

wherein R³, R⁴ and R⁵ are defined as hereinbefore, in the presence of either
a) a secondary amine H-Q, wherein Q denotes —NR^{a}R^{b}, wherein R^{a}, R^{b} independently of one another denote C₁₋₄-alkyl, or —NR^{a}R^{b} denotes pyrrolidinyl, piperidinyl, morpholinyl, piperazinyl or N—C₁₋₄-alkyl-piperazinyl; or
b) an alcohol or thiol H-Q, wherein Q denotes C₁₋₄-alkoxy, C₁₋₄-alkylthio, phenylthio or C₃₋₆-cycloalkyl-oxy, and a secondary amine.

In a sixth aspect the present invention relates to a process for preparing a pyrazole derivative of the formula (III)

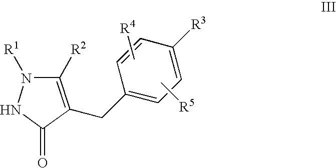

wherein R¹ to R⁵ are defined as hereinbefore;

including the tautomers, stereoisomers, mixtures thereof and the salts thereof; comprising reacting a pyrazole derivative of the formula (XI)

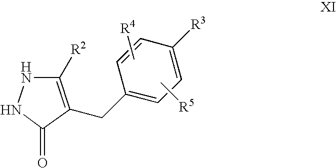

wherein R² to R⁵ are defined as before;

with an alkylating agent R¹—X′ wherein R¹ is defined as before and X′ denotes chlorine, bromine, iodine or C₁₋₃-alkyl-SO₂—O—, in the presence of a base in a solvent or mixture of solvents yielding an intermediate of the formula (XI′)

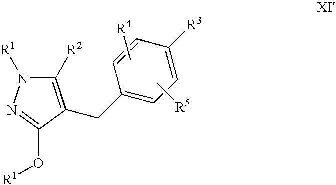

wherein R¹ to R⁵ are defined as hereinbefore; and subsequent cleaving the R¹—O-group at the 3-position of the pyrazole ring in the presence of an acid yielding the aglycone of the formula (III).

In a seventh aspect the present invention relates to a process for preparing a pyrazole derivative of the formula (XI)

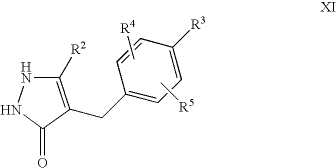

wherein R² to R⁵ are defined as hereinbefore;

including the tautomers, stereoisomers, mixtures thereof and the salts thereof;

comprising the steps:

(i) reacting a benzaldehyde derivative of the formula (V)

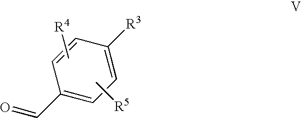

wherein R³, R⁴ and R⁵ are defined as hereinbefore,

with a β-ketoester derivative of the formula (XII)

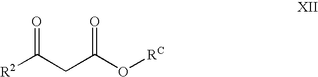

wherein R² is defined as hereinbefore; and R^{C} is methyl, ethyl, n-propyl or i-propyl;

in the presence of an acid and a secondary amine and subsequent or concomitant catalytic hydrogenation; and

(ii) reacting the product of step (i) with hydrazine in a solvent or mixture of solvents.

In a eighth aspect the present invention relates to a process for preparing compounds of the general formula (I) as defined hereinbefore characterized in that the process comprises the process step according to the fifth aspect of the present invention.

In a ninth aspect the present invention relates to a process for preparing compounds of the general formula (I) as defined hereinbefore characterized in that the process comprises one or both process step according to the sixth and/or seventh aspect of the present invention.

In a tenth aspect the present invention relates to a compound of the formula (IV)

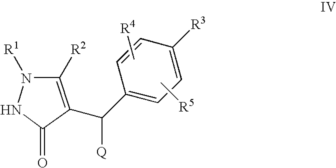

wherein R¹ to R⁵ are defined as hereinbefore, and

Q is Cl, Br, I, C₁₋₄-alkoxy, C₁₋₄-alkylthio, phenylthio, C₃₋₆-cycloalkyl-oxy, C₁₋₄-alkylcarbonyloxy, —NR^{a}R^{b}, wherein R^{a}, R^{b} independently of one another denote C₁₋₄-alkyl, or —NR^{a}R^{b} denotes pyrrolidinyl, piperidinyl, morpholinyl, piperazinyl or N—C₁₋₄-alkyl-piperazinyl, including the tautomers, the stereoisomers, the mixtures thereof and the salts thereof.

In a eleventh aspect the present invention relates to a compound of the formula (III)

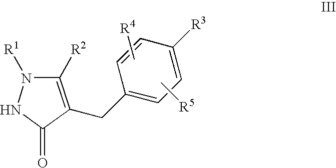

wherein R¹ to R⁵ are defined as in hereinbefore or hereinafter, including the tautomers, the stereoisomers, the mixtures thereof and the salts thereof.

Another aspect of the present invention relates to a compound of the formula (VI)

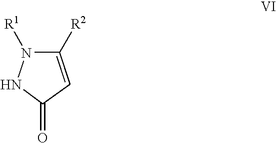

wherein R¹ and R² are defined as in claim 1, 21 or 22, including the tautomers, the mixtures thereof and the salts thereof.

### DETAILED DESCRIPTION OF THE INVENTION

Unless otherwise stated, the groups, residues and substituents, particularly R¹, R², R³, R⁴, R⁵, R⁶, R^{7a}, R^{7b}, R^{7c}, R^{7d}, R¹¹, R¹², R^{C}, Q, X and X′, are defined as above and hereinafter.

If residues, substituents or groups occur several times in a compound, they may have the same or different meanings. For example the meaning di-(C₁₋₄-alkyl)amine encompasses secondary amines with two identical or different alkyl groups, such as ethyl-isopropyl-amine.

In the processes and compounds, intermediates and starting materials according to this invention the following meanings of groups and substituents are preferred:

R¹ preferably denotes a group of the formula

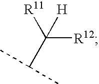

wherein R¹¹ denotes C₁₋₃-alkyl or C₁₋₃-alkyl substituted with one or more fluorine atoms; and R¹² denotes H, or in case R¹¹ denotes methyl, R¹² may also denote a methyl- or ethyl-group or a methyl- or ethyl-group substituted with one or more fluorine atomes; or R¹¹ and R¹² are linked and form together with the CH-group to which they are attached a C₃₋₆-cycloalkyl-group.

Even more preferably R¹ denotes ethyl, n-propyl, i-propyl, cyclobutyl or cyclopentyl; most preferably i-propyl or cyclobutyl.

R² preferably denotes methyl, ethyl, n-propyl or i-propyl; most preferably methyl.

R³ preferably denotes fluorine, chlorine, methyl, methoxy, ethoxy, n-propoxy or i-propoxy; most preferably methyl, methoxy, ethoxy or i-propoxy.

R⁴ preferably denotes fluorine, chlorine, methyl, methoxy, ethoxy, n-propoxy or i-propoxy; in particular fluorine. Furthermore the substituent R⁴ is preferably a substituent in 2-position of the phenyl-ring. Most preferably R⁴ is a substituent in 2-position of the phenyl-ring and denotes fluorine.

R⁵ preferably denotes hydrogen, fluorine, chlorine, methyl or methoxy; most preferably hydrogen or fluorine.

In compounds of the formula (I) the substituents R⁶, R^{7a}, R^{7b}, R^{7c} preferably have independently of one another a meaning selected from the group hydrogen, (C₁₋₄-alkyl)carbonyl, phenylcarbonyl and benzylcarbonyl. Even more preferably in compounds of the formula (I) the substituents R⁶, R^{7a}, R^{7b}, R^{7c} independently of one another have a meaning selected from the group hydrogen, methylcarbonyl and ethylcarbonyl, in particular hydrogen.

In compounds of the formula (II) the substituents R⁶, R⁷ , R^{7b}, R^{7c} preferably have independently of one another a meaning selected from the group (C₁₋₄-alkyl)carbonyl, phenylcarbonyl and benzylcarbonyl. Even more preferably in compounds of the formula (II) the substituents R⁶, R^{7a}, R^{7b}, R^{7c} independently of one another have a meaning selected from the group methylcarbonyl and ethylcarbonyl, in particular methylcarbonyl.

In compounds of the formula (II′) the substituents R⁶ , R^{7a}, R^{7b}, R^{7c}, R^{7d} preferably have independently of one another a meaning selected from the group (C₁₋₄-alkyl)carbonyl, phenylcarbonyl and benzylcarbonyl. Even more preferably in compounds of the formula (II′) the substituents R⁶, R^{7a}, R^{7b}, R^{7c}, R^{7d} independently of one another have a meaning selected from the group methylcarbonyl and ethylcarbonyl, in particular methylcarbonyl.

It is to be understood that the corresponding tautomers of the compounds as specified hereinbefore and hereinafter by general or specific formulae are also included within the scope of any and each process or definition of compound according to this invention. In particular with regard to pyrazole derivatives the following tautomers may exist depending on the reaction and preparation conditions:

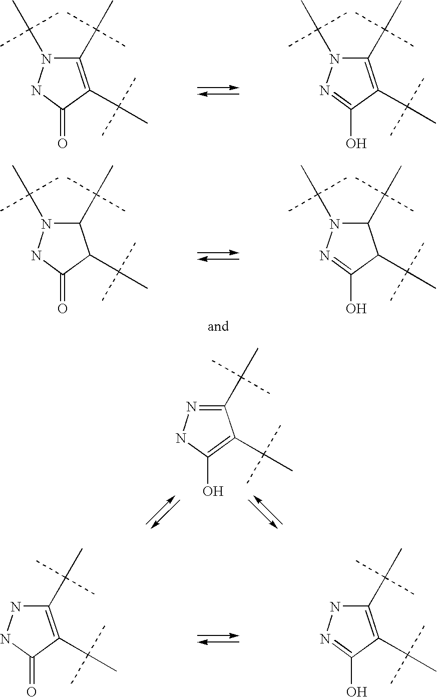

wherein the sign

[symbol]

is used to indicate the bond towards a chemical group or substituent, including hydrogen.

In the following the processes according to this invention are described in detail.

According to the first aspect of the present invention the aglycone of the formula (III) is obtained via catalytic hydrogenation of a compound of the formula (IV) according to the reaction scheme I:

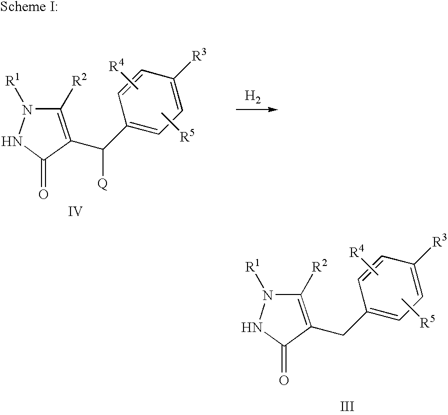

The group Q preferably denotes methoxy, ethoxy, n-propoxy, i-propoxy, —NR^{a}R^{b}, wherein R^{a}, R^{b} independently of one another denote methyl, ethyl, n-propyl or i-propyl, or —NR^{a}R^{b} denotes pyrrolidinyl, piperidinyl, morpholinyl, piperazinyl or N—C₁₋₃-alkyl-piperazinyl. Even more preferably Q denotes methoxy, ethoxy, n-propoxy, i-propoxy, pyrrolidinyl, piperidinyl, morpholinyl, piperazinyl or N—C₁₋₃-alkyl-piperazinyl; most preferably ethoxy, pyrrolidinyl, piperidinyl or morpholinyl.

In the above synthesis step suitable solvents are aliphatic hydrocarbons, aromatic hydrocarbons, alcohols, aliphatic ethers, cyclic ethers, esters, amide type solvents, acetic acid, mixtures thereof and mixtures thereof with water. Examples of suitable solvents are pentane, hexane, benzene, toluene, methanol, ethanol, i-propanol, n-propanol, diethylether, tetrahydrofuran, tetrahydropyran, ethyl acetate, isopropyl acetate, butyl acetate, NMP, DMF, glacial acetic acid, mixtures thereof and mixtures thereof with water. Preferred solvents are methanol, ethanol, i-propanol, n-propanol, tetrahydrofuran, mixtures thereof and mixtures thereof with water.

Preferably the catalytic hydrogenation is carried out in the presence of one or more acids, in particular hydrochloric acid, a carboxylic acid or an alkanesulfonic acid. Examples of suitable acids are hydrochloric acid, acetic acid and trifluoroacetic acid. The acid is preferably taken in an amount equivalent to from about 1 to 150 mol-% relative to the educt of the formula (IV).

In case H-Q is an alcohol or a thiol the acid is preferably taken in an amount equivalent to from about 1 to 50 mol-%, relative to the educt of the formula (UV); even more preferably from about 1 to 20 mol-%, for example about 10 mol-%. In case of acetic acid the amount may even be up to 100 mol-%.

In case Q is selected from —NR^{a}R^{b} according to a preferred embodiment the catalytic hydrogenation may be carried out without the addition of an acid. According to another preferred embodiment in case Q is selected from —NR^{a}R^{b} an acid is preferably taken in an amount equivalent to from about 1 to 120 mol-%, such as for example in an about equimolar amount based on the educt of the formula (IV).

The catalytic hydrogenation is preferably carried out in the presence of a transition metal catalyst such as Pd-based catalysts, for example as finely dispersed Pd, Pd on charcoal or Pd(OH)₂, or Ni-based catalysts, for example as finely dispersed Ni such as Raney-nickel. The suitable amount of catalyst may vary according to the reaction conditions and lies for example in the range from about 0.1 to about 50 weight-%, preferably from about 1 to about 10 weight-% relative to the educt of the formula (IV). The hydrogenation is advantageously carried out at temperatures in the range from −30 to 150° C., preferably from 20 to 100° C., more preferably from 20 to 70° C., most preferably from 40 to 60° C. Suitable hydrogen pressures are usually about equal to or above normal atmospheric pressure, preferably in the range from about 1 to 20 bar, even more preferably from 2 to 8 bar. During the hydrogenation the reaction mixture is preferably agitated or stirred. The time period necessary to complete the hydrogenation can be optimized by experimentation. Usually the hydrogenation is performed in a time period from about 30 min to about 24 hours, preferably from about 1 to 12 hours. After the hydrogenation the catalyst is preferably removed from the reaction mixture, for example by filtration. The next reaction step, i.e. synthesis of a compound of the formula (I), may be carried out with the reaction solution containing the product of the formula (III) or with the isolated product of the formula (III). The product of the formula (III) may be isolated from the reaction solution for example by removing the solvent in vacuum and/or at elevated temperature. The product of the formula (III) may also be obtained by precipitation out of a concentrated reaction solution, for example by adding an antisolvent, such as water, and isolating from the suspension, for example by filtration.

The educt of the formula (IV) wherein Q denotes C₁₋₄-alkoxy, C₁₋₄-alkylthio, phenylthio, C₃₋₆-cycloalkyl-oxy, or —NR^{a}R^{b}, wherein R^{a}, R^{b} independently of one another denote C₁₋₄-alkyl or —NR^{a}R^{b} denotes pyrrolidinyl, piperidinyl, morpholinyl, piperazinyl or N—C₁₋₄-alkyl-piperazinyl, is preferably obtained according to the fifth aspect of the present invention, i.e. by reacting a pyrazole derivative of the formula (VI) with a benzaldehyde derivative of the formula (V) in the presence of either
a) a secondary amine H-Q, wherein Q denotes —NR^{a}R^{b}, wherein R^{a}, R^{b} independently of one another denote C₁₋₄-alkyl, or —NR^{a}R^{b} denotes pyrrolidinyl, piperidinyl, morpholinyl, piperazinyl or N—C₁₋₄-alkyl-piperazinyl; or
b) an alcohol or thiol H-Q, wherein Q denotes C₁₋₄-alkoxy, C₁₋₄-alkylthio, phenylthio or C₃₋₆-cycloalkyl-oxy, and a secondary amine;

according to the reaction scheme II:

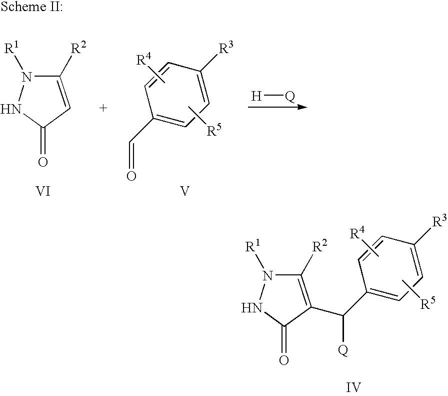

The reaction is carried in the presence of a secondary amine.

In case a compound H-Q is used wherein Q is —NR^{a}R^{b} as defined, then no additional secondary amine is necessary. Preferably H-Q is selected from among pyrrolidine, piperidine, morpholine, piperazine and N—C₁₋₃-alkyl-piperazine; most preferably from among pyrrolidine, piperidine and morpholine. The amine derivative H-Q is preferably employed in an equimolar amount or in a molar excess compared to the pyrazole derivative of the formula (IV). A preferred molar ratio of the amine H-Q to the pyrazole derivative is in the range from about 1:1 to 10:1, even more preferably in the range from 1:1 to 5:1.

In case an alcohol or thiol H-Q is employed wherein Q denotes C₁₋₄-alkoxy, C₁₋₄-alkylthio, phenylthio or C₃₋₆-cycloalkyl-oxy, then the reaction is advantageously carried out in the presence of a secondary amine. Preferably Q is selected from among methoxy, ethoxy, n-propoxy and i-propoxy; most preferably ethoxy. Preferred secondary amines are selected from among di-(C₁₋₄-alkyl)amine or cyclic secondary amines, such as for example pyrrolidin, piperidin, morpholin, piperazin, N—C₁₋₃-alkyl-piperazin. Even more preferably the secondary amine is selected from among pyrrolidine, piperidine, morpholine, piperazine and N—C₁₋₃-alkyl-piperazine; most preferably from among pyrrolidine, piperidine and morpholine. The secondary amine may be used in a catalytic amount, in an about equimolar amount or even in a molar excess. A preferred molar ratio of the secondary amine to the pyrazole derivative is in the range from about 0.05:1 to 2:1, even more preferably from about 0.1:1 to 1.5:1, most preferably from about 1.0:1.0 to 1.5:1.0. The alcohol or thiol H-Q is preferably employed in an equimolar amount or in a molar excess compared to the pyrazole derivative of the formula (VI). In addition to its function as a reaction partner the alcohol H-Q may also serve as a solvent so that in this case molar excesses of H-Q may be used.

Furthermore this reaction according to the present invention is preferably carried out at acidic conditions. Suitable acids are for example C₁₋₆-alkylcarboxylic acids, which may be unsubstituted or substituted with one or more flourine or chlorine substituents, C₁₋₆-alkyl-sulfonic acids, which may be unsubstituted or substituted with one or more flourine or chlorine substituents, dicarboxylic acids, tricarboxylic acids, methylchlorosilanes, non-aqueous mineral acids. Examples of preferred acids are glacial acetic acid, trimethylchlorosilane, hydrochloric acid (aqueous or for example as a solution in an alcohol, such as ethanol), trifluoromethanesulfonic acid. The acid is preferably employed in an equimolar amount or in a molar excess relative to the pyrazole derivative of the formula (IV). A preferred molar ratio of the acid to the pyrazole derivative is in the range from about 0.05:1 to 1:1, even more preferably from about 0.1:1 to 0.5:1.

In case the compound H-Q is an alcohol or thiol derivative as defined above, then the acid is preferably used in at least the molar amount of the secondary amine. In case the group Q is selected from —NR^{a}R^{b}, the reaction may be carried with or without the addition of an acid; whereby preferred acids and molar ratios are given above.

In the above synthesis step suitable solvents are aliphatic hydrocarbons, halogenated aliphatic hydrocarbons, aromatic hydrocarbons, alcohols, aliphatic ethers, cyclic ethers, acetonitril, amide type solvents, acetic acid and mixtures thereof. Examples of suitable solvents are dichloromethane, 1,2-dichloroethane, methanol, ethanol, i-propanol, n-propanol, diethylether, tetrahydrofuran, tetrahydropyran, acetonitril, NMP, DMF, glacial acetic acid and mixtures thereof. Preferred solvents are methanol, ethanol, i-propanol, n-propanol, tetrahydrofuran, acetonitril and mixtures thereof. In case the compound H-Q is an alcohol, it may additionally serve as a solvent and thus may be used in a stoichiometric excess. In case the group Q is selected from —NR^{a}R^{b}, a particularly preferred solvent is acetonitril.

The educts of the formulae (V) and (VI) are preferably reacted in a molar ratio of about 2:1 to 1:2, preverably in an about equimolar ratio. The synthesis step is advantageously carried out at temperatures in the range from −30 to 150° C., preferably from 10 to 100° C., more preferably from 20 to 80° C., most preferably from 30 to 70° C. The time necessary to complete the reaction is usually in the range from about 1 h to 96 h. Depending on the choice of the solvent the product of the formula (IV) is only sparsely soluble in the reaction mixture, thus forming a suspension. The next reaction step, i.e. synthesis of a compound of the formula (III), may be carried out using the crude or the isolated product of the formula (IV).The product of the formula (IV) may be isolated from a reaction solution for example by removing the solvent in vacuum and/or at elevated temperature. The product of the formula (IV) may also be obtained by precipitation out of a concentrated reaction solution or suspension, for example by adding an antisolvent, such as water, and/or cooling of the solution or suspension and isolating from the suspension, for example by filtration.

The synthesis procedures of the benzaldehyde derivatives of the formula (V) are known in the literature or may be carried out in analogy to well-known methods in organic chemistry.

The pyrazole derivative of the formula (VI) is preferably obtained by dehydrogenation of the pyrazole derivative of the formula (VII) according to the reaction scheme III:

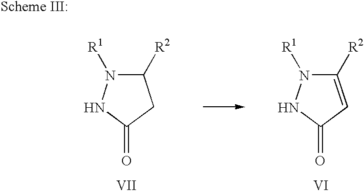

Preferably the dehydrogenation is performed using an oxidizing agent, such as for example H₂O₂, inorganic peroxides, peroxomonosulfuric acid or salts thereof, peroxodisulfuric acid or salts thereof, carboxylic peracids, peroxoborates and the like. A preferred oxidizing agent is H₂O₂ or peracetic acid. H₂O₂ is preferably employed as an aqueous solution, for example with a content of 3 to 90%-weight, preferably 10 to 70%-weight of H₂O₂. The preferred amount of the oxidizing agent is about equimolar or in a molar excess, for example in a molar ratio in the range from 1:1 to 2:1, more preferably from 1.0:1.0 to 1.3:1.0, relative to the educt of the formula (VIII).

According to the above synthesis step the educt of the formula (VII) is dissolved or suspended in a suitable solvent or mixture of solvents. Examples of suitables solvents are carboxylic acids such as for example acetic acid or aqueous mixtures thereof. Due to the exothermic reaction, the oxidizing agent is added to the solution or suspension preferably continuously or in portions over a period of time, for example in the range from 30 min to 24 h. If necessary the reaction mixture may be cooled. The reaction is carried out preferably at temperatures in the range from 0° C. to 130° C., more preferably in the range from 10° C. to 90° C., even more preferably in the range from 20° C. to 80° C. The reaction is usually completed within 1 to 24 h. The final product of the formula (VI) may be obtained in a solid form for example by adding to the reaction mixture water or pouring the reaction mixture into water, preferably in a temperature range from 0 to 20° C., and optionally neutralizing the reaction mixture to a pH in the range from 5 to 9, preferably to a pH of about 7, by using a suitable base, such as for example an aqueous sodium hydroxide, potassium hydroxide or ammonium hydroxide solution, and by removing the solid product from the aqueous reaction mixture, for example by filtration. If the reaction mixture is not neutralized the product may be obtained in a salt form, for example as the acetate.

Alternatively the dehydrogenation is performed catalytically, preferably in the presence of a transition metal catalyst such as Pd-based catalysts, for example as finely dispersed Pd or Pd on charcoal. The catalytic dehydrogenation is preferably carried out at elevated temperatures, for example in the range from about 80° C. to 240° C., preferably from about 100° C. to 200° C. in an chemically inert solvent or solvent mixture, such as aliphatic or aromatic hydrocarbon, for example toluene. The product may be isolated from the reaction, preferably after removing the catalyst, e.g. by filtration, using methods well known in the art.

In cases wherein R¹ denotes a group of the formula

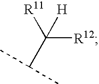

wherein R¹¹ denotes C₁₋₃-alkyl or C₁₋₃-alkyl substituted with one or more fluorine atoms; and R¹² denotes H, or in case R¹¹ denotes methyl, R¹² may also denote a methyl- or ethyl-group or a methyl- or ethyl-group substituted with one or more fluorine atomes; or R¹¹ and R¹² are linked and form together with the CH-group to which they are attached a C₃₋₆-cycloalkyl-group;

the pyrazole derivative of the formula (VII) is preferably obtained by reacting the pyrazole derivative of the formula (VIII) with an aldehyde or ketone of the formula (IX) and subsequent or concomitant reduction according to the reaction scheme IV:

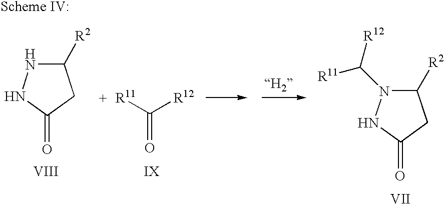

The substituent R¹¹ preferably denotes methyl, ethyl, n-propyl or i-propyl; and the substituent R¹² denotes H; and in case R¹¹ denotes methyl or ethyl, R¹² may also denote methyl or ethyl. R¹¹ and R¹² may be linked and form together with the C-atom to which they are attached a cyclobutyl- or cyclopentyl-ring. Most preferably both substituents R¹¹ and R¹² denote methyl or they are linked to form a cyclobutyl-ring.

In case a salt form of the educt of the formula (VIII) is employed, the neutral form of the formula (VIII) may be obtained by adding a base such as for example sodium hydroxide, potassium hydroxide or ammonium hydroxide, preferably as a solution in an alcohol or water, or an alcoholate, in particular alkali metal alcoholate, such as for example sodium ethanolate in ethanol. The neutralization step and the synthesis step may be performed in situ or the neutral form of the educt of the formula (VIII) may be obtained beforehand.

The above reaction according to the scheme IV is suitably carried out at conditions of reductive aminations which are known to the one skilled in the art.

According to this synthesis step the reactants of the formula (VIII) and (IX) are dissolved or suspended in a suitable solvent or mixture of solvents. Preferred solvents are alcohols, ethers or mixtures thereof with water, such as for example methanol, ethanol, n-propanol, i-propanol, mixtures thereof or mixtures thereof with water. The educt of the formula (IX), such as for example aceton, may serve as a solvent and thus may be employed in a stoichiometric excess.

A preferred molar ratio of the educt of the formula (VIII) and the educt of the formula (IX) is in the range from 1:1 to 1:5, more preferably in the range from 1:1 to 1:3, even more preferably in the range from 1.0:1.5 to 1.0:2.5.

The reduction is preferably performed as a catalytic hydrogenation or alternatively as a reduction using hydrides, in particular borohydrides, such as for example sodium triacetoxyborohydride or sodium cyanoborohydride.

The reaction solution or suspension is catalytically hydrogenated preferably in the presence of a transition metal catalyst such as Pd-based catalysts, for example as finely dispersed Pd or Pd on charcoal. The suitable amount of catalyst may vary according to the reaction conditions and lies for example in the range from 0.1 to 50 weight-%, preferably from 1 to 20 weight-% relative to the educt of the formula (IV). The hydrogenation is advantageously carried out at temperatures in the range from −30 to 150° C., preferably from 20 to 100° C., more preferably from 20 to 80° C., most preferably from 40 to 70° C. Suitable hydrogen pressures are usually about equal to or above normal atmospheric pressure, preferably in the range from about 1 to 20 bar, even more preferably from 2 to 8 bar. During the hydrogenation the reaction mixtures is preferably agitated or stirred. After the hydrogenation the catalyst is preferably removed from the reaction mixture, for example by filtration. The next reaction step, i.e. synthesis of a compound of the formula (VI), may be carried out using with the reaction solution containing the product of the formula (VII) or with the isolated product of the formula (VII). The product of the formula (VII) may be isolated from the reaction solution for example by removing the solvent in vacuum and/or at elevated temperature. In addition the product may be purified via a salt form, for example as its chloride, by adding an acid, such as for example hydrochloric acid in ethanol, to a solution of the product, followed by crystallization, for example supported by cooling and/or inoculating with seed crystalls, and finally isolating the precipitate.

The pyrazole derivative of the formula (VIII) is preferably obtained by reacting an acrylic acid ester derivative of the formula (X) with a hydrazine in a solvent or mixture of solvents according to the reaction scheme V:

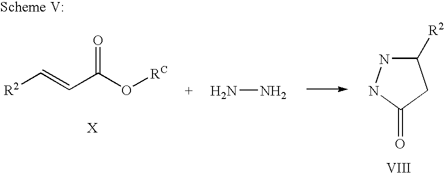

The substituent R^{C} denotes optionally substituted C₁₋₆-alkyl, preferably methyl, ethyl, n-propyl or i-propyl; preferably methyl or ethyl.

In principle this reaction is known to the one skilled in the art. For example the condensation of crotonacidethylester with hydrazin is described by Holan, George et al., Bioorg. Med. Chem. Lett.; 6; 1; 1996; 77-80.

Preferably the acrylic acid ester derivative of the formula (X) is dissolved in a suitable solvent or mixture of solvents such as alcohols, aliphatic ethers, cyclic ethers, and mixtures thereof. Examples of suitable solvents are methanol, ethanol, i-propanol, n-propanol, diethylether, tertbutylmethylether, tetrahydrofuran, tetrahydropyran, dioxane and mixtures thereof or a solution of one or more of these solvents with water. Preferred solvents are ethanol, i-propanol, mixtures thereof or aqueous mixtures thereof.

The hydrazine is advantageously employed as a solution in water, for example as hydrazine monohydrate, or in an alcohol, such as methanol, ethanol, i-propanol, mixtures thereof or mixtures of one or more of such alcohols with water.

A preferred molar ratio of the acrylic acid ester derivative of the formula (X) and of hydrazine is in the range from 1:1 to 1:2, more preferably from 1.0:1.0 to 1.0:1.5, even more preferably in the range from 1.0:1.0 to 1.0:1.2.

Due to the exothermic reaction, the hydrazine is added to the solution of the acrylic acid ester derivative preferably continuously or in portions over a period of time, for example in the range from 15 min to 24 h. If necessary the reaction mixture may be cooled. The reaction is carried out preferably at temperatures in the range from 0° C. to 130° C., more preferably in the range from 20° C. to 100° C., even more preferably in the range from 40° C. to 90° C.

The final product of the formula (VIII) may be obtained by removing the solvents, for example by evaporation in vacuum and/or at elevated temperature. In addition or alternatively the final product may be purified via crystallization in the form of a salt, for example in the form of its hydrohalide, such as hydrochloride. For this purpose an acid, such as for example hydrochloric acid in ethanol or i-propanol, is added to a solution of the product. Crystallization may be supported for example by cooling and/or inoculating with seed crystalls, and finally the precipitate is isolated.

According to a preferred embodiment this reaction step is carried out in an alcohol, in particular in i-propanol, and an aqueous solution of hydrazine, in particular hydrazine hydrate is used. After or during the reaction water is preferably removed from the reaction mixture by azeotropic destillation. The product is advantageously obtained as described above, in particular by precipitating a salt form, preferably the chloride, for example by adding a solution of hydrochloric acid in ethanol.

The compound of the formula (I) is preferably obtained by reacting the aglycone of the formula (III) as described hereinbefore with a glucose derivative of the formula (II) according to the reaction scheme VIa:

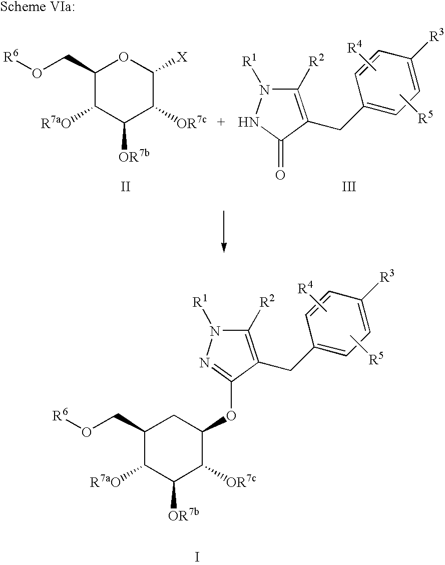

Preferably in the compounds of the formula (II) and (I) the substituents R⁶, R^{7a}, R^{7b}, R^{7c} are independently of one another selected from the group consisting of (C₁₋₄-alkyl)carbonyl, phenylcarbonyl and benzylcarbonyl. Even more preferably the substituents R⁶, R⁷ , R^{7b}, R^{7c} independently of one another have a meaning selected from the group methylcarbonyl and ethylcarbonyl, in particular methylcarbonyl.

The substituent X preferably denotes a bromine atom. The substituents R¹ to R⁵ are defined as hereinbefore.

According to a first embodiment this process step may be carried out in a solvent or a mixture of solvents which exhibits sufficient solubility properties in view of the starting materials of the formulae (II) and (III) and in the presence of a suitable base. Preferred organic solvents with such properties are ketones, ethers, cyclic ethers, acetonitril, and mixtures thereof. Examples of preferred organic solvents are acetone, methyl ethyl ketone, diethyl ketone, cyclohexanone, cyclopentanone, acetonitril, THF, NMP, DMF, and mixtures thereof. Suitable bases are in particular carbonates, such as for example sodium carbonate, potassium carbonate, silver carbonate or cadmium carbonate.

According to a preferred second embodiment this process step is carried out in a reaction mixture comprising two liquid phases, preferably under phase-transfer conditions. Advantageously a biphasic solvent system and one or more phase-transfer catalysts are used. Preferred solvents of the first phase are aprotic organic solvents, in particular aromatic hydrocarbons (for example benzene, chlorobenzene, trifluoromethylbenzene, toluene, xylenes), alkanes (for example pentane, hexane, heptane, octane), halogenated alkanes (for example CH₂Cl₂, CHCl₃, ClCh₂CH₂Cl), ethers (for example 2-methyl-tetrahydrofuran), esters (for example isopropylacetate), and mixtures thereof. A particularly preferred solvent of the first phase comprises a chlorinated C₁₋₃-alkane which additionally may have one or more fluorine substituents, most preferably CH₂Cl₂.

The second phase is preferably water or an aqueous mixture of a protic solvent. The most preferred solvent of the second phase is water.

A preferred ratio of the volume of the first solvent phase to the volume of the second phase is in the range from 1: 10 to 10: 1, even more preferably in the range from 1:5 to 5:1, most preferably in the range from 1:5 to 2:1.

Preferred phase-transfer catalysts possess a quarternary ammonium cation, as for example tetraalkylammonium, N-aryl-N-trialkylammonium, N-arylalkyl-N-trialkyl-ammonium compounds, wherein the alkyl-residues may be identical or different. Examples are tetramethylammonium compounds, tetraethylammonium compounds, tetrabutylammonium or benzyl trimethyl ammonium compounds. Most preferred phase-transfer catalyst are tetrabutylammonium compounds, in particular tetrabutylammonium salts with inorganic acids, such as tetrabutylammonium chloride, tetrabutylammonium bromide, tetrabutylammonium hydrogensulfate, etc.

The preferred amount of the phase-transfer catalyst depends on the kind of solvents used and their quantities and can be determined by standard experimentation. Usually per 1 mole of the starting material of the formula (III) an amount from 0.01 to 1.0 mol, even more preferably from 0.02 to 0.5, such as for example about 0.05 mol of a phase-transfer catalyst is used.

Advantageously the second solvent phase is basified or buffered. During and after the reaction, preferred pH-values of an aqueous solvent phase are greater than or equal to about 10, in particular greater than or equal to about 11, even more preferably greater than or equal to about 12, most preferably in the range from about 11 to about 15, most preferably in the range from about 12 to about 14.

The pH value is advantageously kept in the desired basic pH range advantageously by the addition of at least one basifying reagent, preferably selected from the group consisting of hydroxides, carbonates, phosphates and/or borates. The corresponding alkali salts are preferred, as for example sodium carbonate, sodium hydroxide, potassium carbonate, potassium hydroxide and/or sodium borate. Advantageously the basifying reagent is added in the form of an aqueous solution; for example as an aqueous sodium or potassium hydroxide solution.

A preferred molar ratio of the educt of the formula (II) and the educt of the formula (III) is in the range from 5:1 to 1:2, more preferably in the range from 3:1 to 1:1, even more preferably in the range from 2.0:1.0 to 1.0:1.0.

A preferred temperature range for the reaction of the compound (II) with the compound of the formula (III) is from about 0° C. to 50° C., even more preferably in the range from about 5° C. to 45° C., most preferably in the range from about 15° C. to 40° C. As the reaction is exothermic cooling of the reaction mixture might become necessary.

Depending on the reaction conditions the reaction is carried out usually in a time period from 30 min to 48 hours, preferably from 2 to 24 hours.

An end point of the reaction may be detected by the amount of remaining compound of the formula (III), for example by HPLC.

The compound of the formula (I) may be isolated from the reaction mixture by methods well known to the one skilled in the art. For example if the reaction was carried out under phase transfer conditions involving an aqueous and an organic phase, the aqueous phase is separated and extracted with an organic solvent or mixture of organic solvents; the organic phases are combined and washed with water or an aqueous solution, preferably with an acidic aqueous solution, and finally dried and the solvents are removed by evaporation in reduced pressure and/or at elevated temperature to yield the compound of the formula (I). In the following reaction step according to the third aspect of the present invention the compound of the formula (I), wherein one or more of the substituents R⁶ , R⁷ , R^{7b}, R^{7c} are not hydrogen, is deprotected to yield the final product of the formula (IH) according to the reaction scheme VIb:

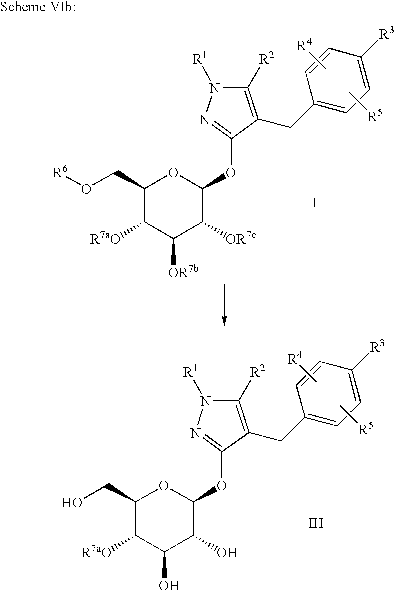

Preferably this reaction step is carried out using the crude product or the reaction mixture of the previous reaction step. Alternatively the isolated and optionally purified product of the formula (I) is used.

Suitable methods for deprotection are well-known to the one skilled in the art. For example acyl protecting groups may be cleaved hydrolytically in an aqueous solvent, e.g. in water, isopropanol/water, acetic acid/water, tetrahydrofuran/water or dioxane/water, in the presence of an acid such as trifluoroacetic acid, hydrochloric acid or sulphuric acid.

Preferably acyl protecting groups are cleaved using an alcoholate, in particular C₁₋₄-alcoholate, for example sodium ethanolate or potassium-t-butoxide in ethanol, whereby the absence of water is preferred. Suitable solvents are alcohols, such as methanol, ethanol or n-propanol. As this deprotection is preferably carried out as a transesterefication, advantageously only catalytical amounts of the alcoholate, preferably from about 0.1 to 50 mol-%, even more preferably from about 1 to 20 mol-% relative to the educt of the formula (I) are needed. Suitable temperatures are between 0° C. and the boiling point of the reaction mixture, preferably between 5 and 40° C. The reaction is usually completed within 1 to 48 h. After completion of the reaction the reaction mixture is preferably neutralized or slightly acidified, for example by using acetic acid, and solvents may be removed by destillation under reduced pressure and/or elevated temperatures. The product of the formula (IH) is obtained as a resinous solid.

The glucose derivative of the formula (II) wherein X denotes a chlorine or a bromine atom may be obtained by methods known to the one skilled in the art and described in the literature. Preferably the glucose derivative of the formula (II) is obtained by reacting a protected glucose derivative of the formula (II′) with HBr in a solvent or mixture of solvents according to the reaction scheme VII.

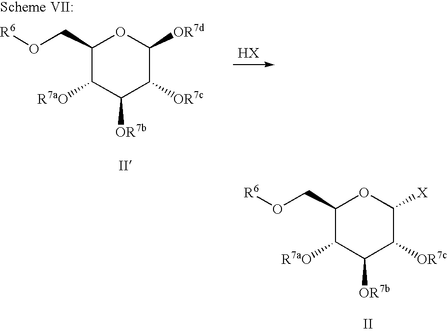

Preferably in the compounds of the formula (II) and (I) the substituents R⁶, R^{7a}, R^{7b}, R^{7c} and R^{7d}, where applicable, are independently of one another selected from the group consisting of (C₁₋₄-alkyl)carbonyl. Even more preferably the substituents R⁶, R⁷, R^{7b}, R^{7c} and R^{7d}, where applicable, independently of one another have a meaning selected from the group methylcarbonyl and ethylcarbonyl, in particular methylcarbonyl.

The substituent X preferably denotes bromine.

This reaction step is preferably carried out in a solvent or a mixture of solvents. Suitable solvents are preferably aliphatic hydrocarbons, halogenated aliphatic hydrocarbons, aromatic hydrocarbons and mixtures thereof. Examples of suitable solvents are pentane, hexane, dichloromethane, 1,2-dichloroethane, benzene, toluene, xylenes, and mixtures thereof. Preferred solvents are dichloromethane, benzene, toluene, xylenes or mixtures thereof, in particular dichloromethane or toluene.

According to a preferred embodiment of this reaction step the starting material of the formula (II′) is dissolved or suspended in the solvent or mixture of solvents and HX or a solution of HX is added. In case HX is HBr a preferred solution is HBr in acetic acid, for example a 30% solution of HBr in acetic acid. A suitable amount of HX is about equimolar or in a molar excess relative to the protected glucose of the formula (II′). Preferably the molar ratio of HX to the glucose derivative of the formula (II′) is in the range from about 1:1 to 10:1; even more preferably in the range from about 2:1 to 6:1.

In order to remove any amounts of water in the reaction mixture, so as for example a content of water of a hygroscopic solution of HX, it is advantageous to add a compound which chemically binds or removes water, such as acetic anhydride.

The reaction is preferably carried out at temperatures in a range from 0° C. to 40° C., most preferably from 10° C. to 30° C. Usually the reaction is completed in a period of time from 10 min to 12 hours.

The product of the formula (II) may be isolated from the reaction mixture by methods well known in the art. For example in case of a solvent or mixture of solvents not or only slightly miscible with water, the reaction mixture may be washed with water and/or a saturated sodium chloride solution; any excess of acids may be neutralized, in particular by washing the combined organic phases with a basic aqueous solution, such as for example an aqueous saturated sodium hydrogencarbonate solution; and finally the solvents may be evaporated in vacuum. The product may be isolated and purified by crystallization, preferably by using a suitable solvent, for example by solving the product in tertbutylmethylether and then adding methylcyclohexane.

Alternatively the following reaction step according to the scheme Via is carried out using the crude product of the formula (II), which is advantageously neutralized beforehand. For example the reaction mixture is washed with an aqueous basic solution and the neutralized organic phase is dried.

In an alternative embodiment corresponding to the sixth aspect of the present invention the pyrazole derivative of the formula (III) may be obtained by reacting a pyrazole derivative of the formula (XI) with an alkylating agent R¹—X′ wherein R¹ is defined as hereinbefore and X′ denotes chlorine, bromine, iodine or C₁₋₃-alkyl-SO₂—O—, in the presence of a base in a solvent or mixture of solvents yielding an intermediate of the formula (XI′) and subsequent cleaving the R¹—O-group at the 3-position of the pyrazole ring, in particular in the presence of an acid, yielding the aglycone of the formula (III) according to the reaction scheme VIII:

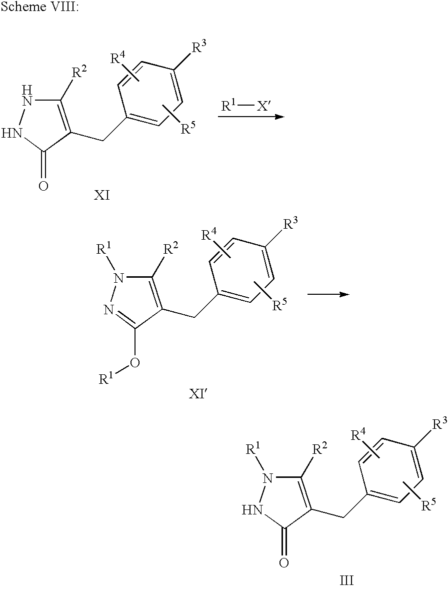

In the formulae of the Scheme VIII the substituents R¹ to R⁵ are defined as hereinbefore. Preferably R¹ denotes methyl, ethyl, n-propyl, i-propyl, cyclobutyl or cyclopentyl; most preferably i-propyl or cyclobutyl.

The group X′ is preferably bromine.

The pyrazole derivative of the formula (XI) is reacted with an alkylating agent R¹-X′ in the presence of a base, preferably a strong base. Suitable strong bases are selected from the group consisting of alkali hydroxides, alcoholates, hydrides. Examples of preferred strong bases are sodium hydroxide and potassium hydroxide.

As the O-atom of the pyrazolon-group is also at least partially alkylated, advantageously a molar excess of the alkylating agent is employed. A preferred molar ratio of the alkylating agent compared with the educt of the formula (XI) is above about 2:1, even more preferably in the range from 2:1 to 8:1; for example in the range from 3:1 to 5:1.

The molar amount of the base advantageously employed is about the same as the amount of the alkylating agent. Therefore advantageously a molar excess of the base is taken. A preferred molar ratio of the base compared with the educt of the formula (XI) is above about 2:1, even more preferably in the range from 2:1 to 8:1; for example in the range from 3 1 to 5:1.

According to the above alkylation step the educt of the formula (XI) and the strong base are dissolved or suspended in a suitable solvent or mixture of solvents. Suitable solvents are polar solvents or mixtures thereof which exhibit sufficient solubility properties in view of the starting material (XI), the alkylating agent R¹—X′ and the base. Suitable solvents may be selected from the group consisting of aliphatic ethers, cyclic ethers, amide type solvents, and mixtures thereof. Examples of preferred solvents are NMP, DMF, DMA, and mixtures thereof.

Then the alkylating agent or a solution thereof is preferably added to the reaction mixture at once or advantageously over a period time, for example within a period from 5 min to 4 hours. The reaction is preferably carried out at temperatures in a range from −20° C. to 50° C., even more preferably from −10° C. to 40° C., most preferably from 5° C. to 35° C. An end point of the reaction may be detected for example by thin layer chromatography or HPLC. Depending on the reaction conditions the reaction is usually carried out in a time period from 30 min to 48 hours, preferably from 2 to 24 hours.

The product of the formula (XI′) may be obtained from the reaction mixture by methods well-known to the one skilled in the art. A further purification is usually not necessary. In particular in case the reaction yields any by-products, such as for example derivatives which are alkylated in the 2-position of the pyrazole ring, such by-products do not necessarily need to be removed at the end of this reaction step. According to an example how to obtain the reaction product the reaction mixture is poured onto cold water and an organic solvent, such as an aliphatic or aromatic hydrocarbon, for example toluene, is added. The aqueous phase is neutralized or slightly acidified using an acid, such as concentrated hydrochloric acid. The organic phase is separated and optionally the aqueous phase is extracted again with organic solvents. The combined organic phases may be washed with water and/or saturated aqueous sodium chloride solution and dried. The product of the formula (XI′) may be obtained by removing the organic solvents, preferably in vacuum and/or at elevated temperatures.

In order to obtain the aglycone of the formula (III) the substituent R¹—O— has to be cleaved at the 3-position of the pyrazole ring of the compound (XI′). Preferably the cleavage is done in the presence of an acid, more preferably in the presence of a strong acid, such as HCl, HBr, Hl, H₂SO₄, or alkylsulfonic acids, such as for example methanesulfonic acid, which is added for example as aqueous solution.

Advantageously a molar excess of the acid is employed. A preferred molar ratio of the acid compared with the educt of the formula (XI′) is above about 2:1, even more preferably in the range from 2:1 to 40:1; for example in the range from 4:1 to 20:1.

The educt of the formula (XI′) and the acid are dissolved or suspended in a suitable solvent or mixture of solvents. Suitable solvents are for example water, alcohols, carboxylic acids, and mixtures thereof; in particular water, ethanol, acetic acid. Preferably the acid is used in the form of a solution in water, an alcohol or a mixture thereof; in this case the acidic solution may serve as a solvent so that less or no additional solvent may be needed.

The reaction is preferably carried out at temperatures in a range from 40° C. to 180° C., even more preferably from 60° C. to 160° C., most preferably from 80° C. to 160° C. The reaction is preferably carried out in a closed reactor or autoclave.

An end point of the reaction may be detected for example by thin layer chromatography or HPLC. Depending on the reaction conditions the reaction is carried out usually in a time period from 15 min to 24 hours, preferably from 1 to 12 hours.

The product may be obtained via crystallization from the reaction mixture. Preferably the type and the amount of the solvent is chosen such that the reactants are dissolved at the reaction temperatures. Then after cooling of the reaction mixture the product may precipitate whereby additional measures to start crystallization such as inoculating with seed-crystalls and/or adding an antisolvent may be employed. The crystalls may be isolated for example by filtration and washed in a suitable solvent such as an alcohol, for example iso-propanol, and optionally dried thereafter.

According to the seventh aspect of the present invention the pyrazole derivative of the formula (XI) may be obtained by

(i) reacting a benzaldehyde derivative of the formula (V) with a β-ketoester derivative of the formula (XII) in the presence of an acid and a secondary amine and subsequent or concomitant catalytic hydrogenation; and

(ii) reacting the product of step (i) with hydrazine in a solvent or mixture of solvents:

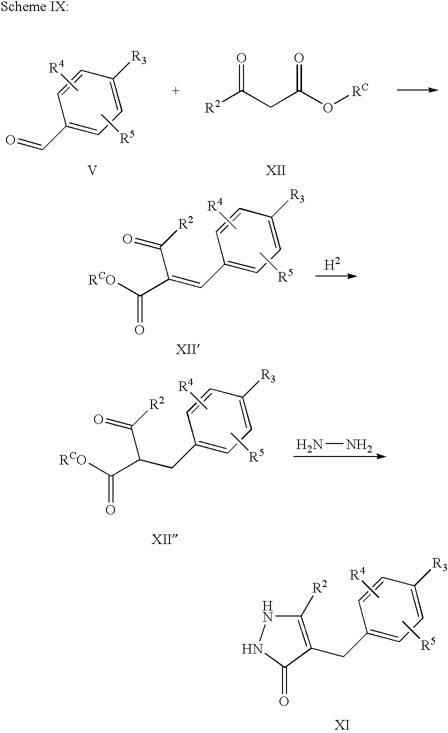

The group R^{C} denotes methyl, ethyl, n-propyl or i-propyl; preferably methyl or ethyl.

The group R² preferably denotes methyl, ethyl, n-propyl or i-propyl; most preferably methyl.

A preferred compound of the formula XII is methylacetoacetate.

In the first reaction step according to the above scheme the benzaldehyde derivative of the formula (V) is reacted with a -ketoester derivative of the formula (XII) in the presence of an acid and a secondary amine.

This reaction step is suitably carried at conditions of Knoevenagel reactions which are known to the one skilled in the art.

The molar ratio of the benzaldehyde of the formula (V) to the β-ketoester of the formula (XII) is preferably in the range from about 2:1 to 1:2, more preferably from about 1.3:1 to 1:1.3, in particular equimolar.

Suitable acids are carboxylic acids, and mixtures thereof, such as for example acetic acid.

The molar ratio of the acid to the benzaldehyde derivative of the formula (V) is preferably from about 2:1 to about 0.8:1, more preferably from about 1.5:1 to about 1:1. Most preferably about equimolar amounts of the acid and the benzaldehyde derivative are taken.

Suitable secondary amines are di(C₁₋₄-alkyl)amines, saturated or unsatured heterocycles with at least one secondary amine group, such as for example dimethylamine, ethylmethylamine, diethylamine, di(isopropyl)amine, pyrrolidine, piperidine, piperazine, morpholine, N—(C₁₋₃-alkyl)piperazine, and mixtures thereof. Preferred amines are piperidine and pyrrolidine.

The molar ratio of the secondary amine to the benzaldehyde derivative of the formula (V) is preferably from about 0.05:1 to about 1:1, more preferably from about 0.1:1 to about 0.7:1, most preferably from about 0.15:1 to about 0.5:1.

The reaction may be carried without an additional solvent or in a solvent or a mixture of solvents. Suitable solvents are aromatic solvents, ethers, alkanes, cycloalkanes, alcohols, or mixtures thereof. According to a preferred embodiment no additional solvent is used.

According to a preferred embodiment of this reaction step the benzaldehyde derivative of the formula (V), the P-ketoester derivative of the formula (XII) and the acid are mixed; optionally with one or more solvents. The secondary amine is added to the reaction mixture whereby if needed the reaction mixture may be cooled.

Preferably the reaction is carried out at temperatures in the range from −10° C. to 80° C., more preferably from 0° C. to 60° C., even more preferably from 10° C. to 40° C.

In order to complete the reaction usually a time period from 30 min to 48 hours, in particular from 2 to 24 hours is needed.

The intermediate of the formula (XII′) may be isolated and, if needed, purified using methods well known in the art.

In the following reaction step the intermediate of the formula (XII′) is catalytically hydrogenated. Preferably the hydrogenation is carried out with the raw product of the previous reaction step, for example using the reaction mixture of the previous reaction step. The catalytic hydrogenation may be carried out after the completion of the first reaction step or concomitantly to the first reaction step, i.e. when the reaction according to the first reaction step is not completed, as during the hydrogenation a reaction according to the first reaction step still may take place.

With regard to the step of catalytical hydrogenation those solvent or mixtures thereof can be taken which are described as suitable or preferred according to the previous synthesis step. In general suitable solvents are aliphatic hydrocarbons, aromatic hydrocarbons, alcohols, aliphatic ethers, cyclic ethers, and mixtures thereof. Examples of suitable solvents are pentane, hexane, benzene, toluene, methanol, ethanol, i-propanol, n-propanol, diethylether, tetrahydrofuran, tetrahydropyran and mixtures thereof. Preferred solvents are methanol, ethanol, i-propanol, n-propanol, tetrahydrofuran and mixtures thereof. In case in the previous reaction step no additional solvent was used, preferably one or more solvents are added before the catalytic hydrogenation is performed.

The catalytic hydrogenation is preferably carried out in the presence of a transition metal catalyst such as Pd-based catalysts, for example as finely dispersed Pd or Pd on charcoal, or Ni-based catalysts, for example as finely dispersed Ni such as Raney-nickel. The suitable amount of catalyst may vary according to the reaction conditions and lies for example in the range from about 0.1 to about 50 weight-%, preferably from about 1 to about 10 weight-% relative to the educt of the formula (V) or to the intermediate of the formula (XII′).

Therefore at the end of the previous synthesis step or—if the hydrogenation is to be carried out concomitantly to the first reaction step—during or at the beginning of the previous synthesis step the appropriate amount of the catalyst and optionally additional solvent or mixture of solvents are added to the reaction mixture. Alternatively the isolated intermediate of the formula (XII′) is dissolved in the solvent or mixture of solvents and the catalyst is added thereto.

The hydrogenation is advantageously carried out at temperatures in the range from −10 to 150° C., preferably from 20 to 100° C., more preferably from 20 to 80° C., most preferably from 40 to 70° C. Suitable hydrogen pressures are usually about equal to or above normal atmospheric pressure, preferably in the range from about 1 to 20 bar, even more preferably from 2 to 8 bar. During the hydrogenation the reaction mixtures is preferably agitated or stirred. The time period necessary to complete the hydrogenation can be optimized by experimentation. Usually the hydrogenation is performed in a time period from about 30 min to about 24 hours, preferably from about 1 to 12 hours. After the hydrogenation the catalyst is preferably removed from the reaction mixture, for example by filtration.

The product of the hydrogenation of the formula (XII′) may be isolated and, if needed, purified using methods well known in the art.

In the following final reaction step the intermediate of the formula (XII″) is reacted with hydrazine to yield the product of the formula (XI). Preferably this reaction step is carried out with the raw product, for example using the reaction mixture of the previous reaction step; preferably after the catalyst has been removed from the reaction mixture. Alternatively the isolated product of the formula (XII″) is used.

Suitable solvent or mixture of solvents of this reaction step are alcohols, aliphatic ethers, cyclic ethers, mixtures thereof and mixtures of one or more of these solvents with water. Examples of suitable solvents are methanol, ethanol, i-propanol, n-propanol, diethylether, tertbutylmethylether, tetrahydrofuran, tetrahydropyran, mixtures thereof or a solution of one or more of these solvents with water. A preferred solvent is the solvent as used in the previous reaction, in particular isopropanol. In case the reaction mixture of the previous reaction step, i.e. not the isolated product of the formula (XII″), is taken, no additional solvent may be needed.

The hydrazine is advantageously employed as a solution in water, for example as hydrazine monohydrate, or in an alcohol, such as methanol, ethanol, i-propanol, mixtures thereof or mixtures of one or more of such alcohols with water.

A preferred molar ratio of the intermediate of the formula (XII″) or relative to the educt of the formula (V) and of hydrazine is in the range from 1:1 to 1:2, more preferably from 1.0:1.0 to 1.0:1.5, even more preferably in the range from 1.0:1.0 to 1.0:1.2.

Therefore at the end of the previous synthesis step the appropriate amount of hydrazine and optionally additional solvent or mixture of solvents are added to the reaction mixture. Alternatively the isolated intermediate of the formula (XII″) is dissolved in the solvent or mixture of solvents and the hydrazine is added thereto.

Due to the exothermic reaction, the addition of hydrazine is preferably done continuously or in portions over a period of time, for example in the range from 30 min to 24 h. If necessary the reaction mixture may be cooled. The reaction is carried out preferably at temperatures in the range from 0° C. to 140° C., more preferably in the range from 20° C. to 110° C., even more preferably in the range from 40° C. to 90° C. It may be advantageous to complete the reaction at lower temperatures, for example in the range from 15° C. to 40° C., for an additional period of time, for example from 1 to 24 hours.

The final product of the formula (XI) is advantageously obtained as a solid or a precipitate out of the reaction mixture or suspension by isolating it from the liquid phase, for example by filtration. The solid may be purified, for example by washing in a suitable solvent, such as those described at the beginning of this reaction step. Alternatively the product may be obtained by removing the solvents, for example by evaporation in vacuum and/or at elevated temperature. In addition or alternatively the final product may be purified via crystallization.

All reactions according to this invention, but not those involving hydrogenation, are preferably carried out at normal atmospheric pressure. As these process steps are not sensitive to the pressure, they can be carried out at slightly reduced pressure or at an elevated pressure also.

Furthermore preferably all reaction steps according to this invention are carried out under an inert atmosphere, for example in nitrogen or argon.

Preferred compounds of the formula VI are selected from the formulae VI.1 and VI.2

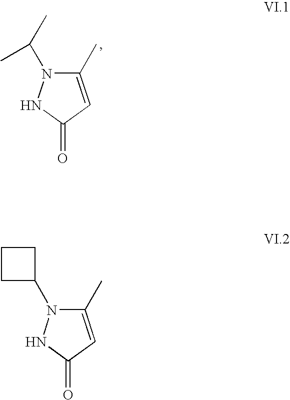

including the tautomers and mixtures thereof.

The present invention also relates to a compound of the formula (IV)

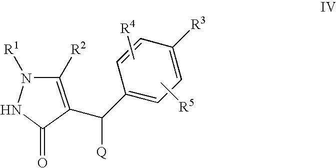

wherein R¹ to R⁵ and Q are defined as hereinbefore.

R¹ preferably denotes methyl, ethyl, n-propyl, i-propyl, cyclobutyl or cyclopentyl; most preferably i-propyl or cyclobutyl.

R² preferably denotes methyl, ethyl, n-propyl or i-propyl; most preferably methyl.

R³ preferably denotes fluorine, chlorine, methyl, methoxy, ethoxy, n-propoxy or i-propoxy; most preferably methyl, methoxy, ethoxy or i-propoxy;

R⁴ preferably denotes fluorine, chlorine, methyl, methoxy, ethoxy, n-propoxy or i-propoxy; most preferably fluorine. Furthermore R⁴ is preferably a substituent in 2-position of the phenyl-ring, i.e. in meta-position relative to R³.

R⁵ preferably denotes hydrogen, fluorine, chlorine, methyl or methoxy; most preferably hydrogen or fluorine.

The group Q preferably denotes methoxy, ethoxy, n-propoxy, i-propoxy, —NR^{a}R^{b}, wherein R^{a}, R^{b} independently of one another denote methyl, ethyl, n-propyl or i-propyl, or —NR^{a}R^{b} denotes pyrrolidinyl, piperidinyl, morpholinyl, piperazinyl or N—C₁₋₃-alkyl-piperazinyl. Even more preferably Q denotes methoxy, ethoxy, n-propoxy, i-propoxy, pyrrolidinyl, piperidinyl, morpholinyl, piperazinyl or N—C₁₋₃-alkyl-piperazinyl; most preferably ethoxy, pyrrolidinyl, piperidinyl or morpholinyl.

Therefore according to this invention compounds of the formula (IV.1) to (IV.11) as listed in the following table are preferred:

| | |
|---|---|
| 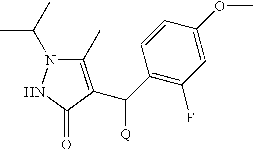 | IV.1 |
| 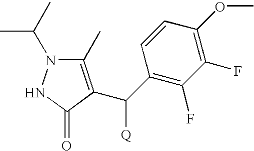 | IV.2 |
| 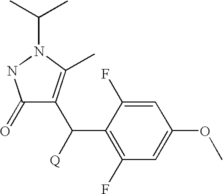 | IV.3 |
| 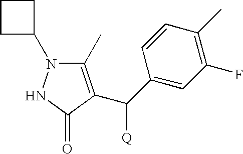 | IV.4 |
| 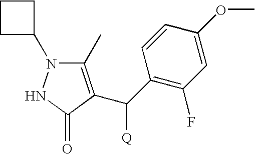 | IV.5 |
| 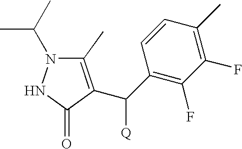 | IV.6 |
| 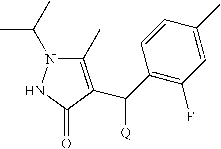 | IV.7 |
| 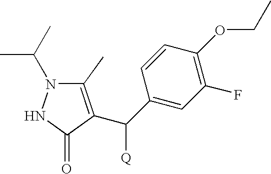 | IV.8 |
| 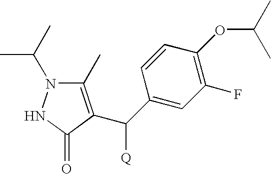 | IV.9 |
| 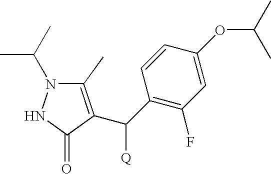 | IV.10 |
| 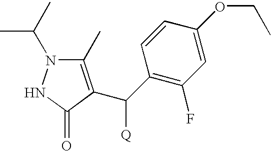 | IV.11 |

in which Q is defined as hereinbefore and hereinafter.

In particular those compounds of the formulae (IV.1) to (IV.11) are preferrred in which Q is selected from methoxy, ethoxy, iso-propoxy, pyrrolidinyl, piperidinyl, morpholinyl or methylcarbonyloxy. Most preferably in the above compounds of the formulae (IV.1) to (IV.11) Q denotes ethoxy.

Furthermore according to this invention compounds of the formula (III.1) to (III.11) as listed in the following table are preferred:

| | |
|---|---|
| 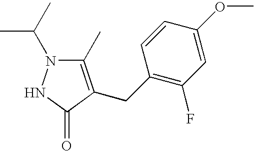 | III.1 |
| 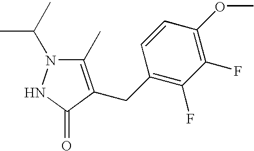 | III.2 |
| 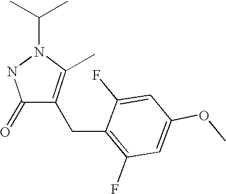 | III.3 |
| 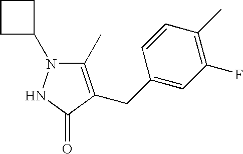 | III.4 |
| 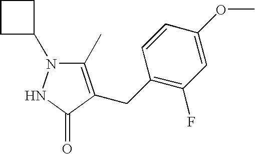 | III.5 |
| 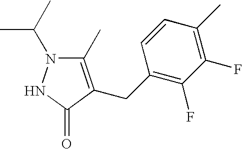 | III.6 |
| 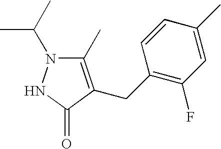 | III.7 |
| 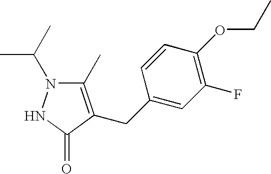 | III.8 |
| 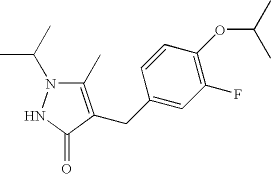 | III.9 |
| 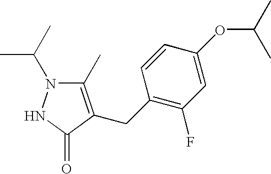 | III.10 |
| 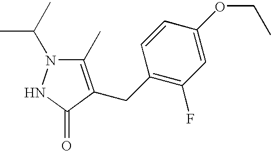 | III.11 |

In addition compounds of the formulae (I.1), (I.2), (I.3), (I.4), (I.5), (I.6), (I.7), (I.8), (I.9), (I.10), (I.11) as they are depicted in the experimental section, including the tautomers, the stereoisomers, the mixtures thereof and the salts thereof, are preferred.

The compounds of the formula (I), in particular of the formula (IH), including prodrugs thereof and pharmaceutically acceptable salts, show activity as inducers of urinary sugar excretion and thus may be used in the manufacture of medicaments in the treatment of diabetes.

In the foregoing and following text, H atoms of hydroxyl groups are not explicitly shown in every case in structural formulae. The Examples that follow are intended to illustrate the present invention without restricting it. In case the pressure is indicated in the unit “bar”, the corresponding values can be converted into SI units by using 1 bar=0.1 MPa. In case the pressure is indicated in the unit “psi”, the corresponding values can be converted into SI units by using 1 psi=6894.757 Pa. The following abbreviations are used hereinbefore and hereinafter:

| | |
|---|---|
| DMA | dimethylacetamide |
| DMF | dimethylformamide, |
| NMP | N-methyl-2-pyrrolidone, |
| THF | tetrahydrofuran. |

Experimental Procedures:

### EXAMPLE 1

### Synthesis of Preparation of 1,2-dihydro-1-(1-methylethyl)-5-methyl-3H-pyrazol-3-one (VI.1)

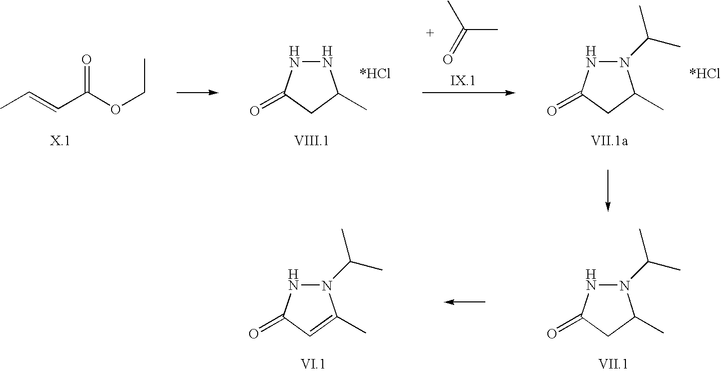

### EXAMPLE 1.1

### Preparation of 5-Methyl-3-pyrazolidinone monohydrochloride (VIII.1)

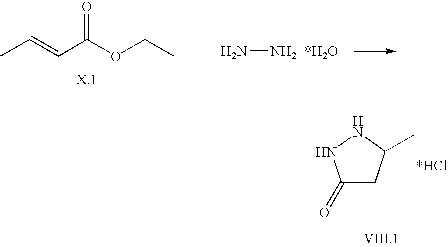

Ethyl crotonate (500 ml; 3.94 mol) is dissolved in isopropanol (1.85 L) and heated to 50° C. Hydrazine hydrate (215 ml; 4.34 mol) is added within 30 min. and the reaction mixture is heated to reflux for 2 h. The solvent is then distilled off (approx. 1 L) under reduced pressure. Isopropanol (400 ml) is then added and the reaction mixture is cooled to 22° C. Hydrochloric acid 11.7 N in ethanol (375 ml, 3.94 mol) is added and the reaction mixture is stirred at about 20 to 25° C. 15 h. The reaction mixture is then cooled to 0° C., filtered and the product is washed with isopropanol (3 times with each 200 ml); then dried to constant weight at 45° C. to yield colorless crystals. Mass and ¹H-NMR-spectra are in accordance with the assigned structure.

Mass spectrum (ESI⁺): m/z=101 [M+H]⁺

### EXAMPLE 1.2

### Preparation of 1-(1-Methylethyl)-5-methyl-3-pyrazolidinone monohydrochloride (VII.1a)

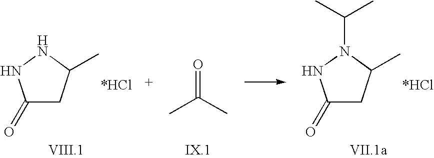

5-Methyl-3-pyrazolidinone monohydrochloride (692 g; 5.07 mol) is suspended in isopropanol (4.9 L). Aqueous 50% sodium hydroxide (270 ml; 5.07 mol) and palladium (10%-weight) on charcoal (70 g) together with acetone (744 ml, 10 mol) are added. The mixture is then hydrogenated under an atmosphere of hydrogen at 50° C. 3 bar (42 psi) until uptake of hydrogen ceases. The reaction mixture is filtered and solvent is distilled off under reduced pressure.

The residue is treated two times with 1 L of isopropanol which is subsequently distilled off under reduced pressure. The remainder is dissolved in isopropanol (3.5 L) and filtered. To the filtrate is added hydrogen chloride 10.5N in ethanol ( 482 ml; 5.06 mol) which causes the precipitation of the hydrochloride salt which is isolated by filtration. It is washed two times with isopropanol ( 2×500ml ) and dried at 45° C. to yield the product as colourless crystals.

Mass and ¹H-NMR-spectra are in accordance with the assigned structure.

Mass spectrum (ESI⁺): m/z=143 [M+H]⁺

### EXAMPLE 1.3

### Preparation of 1-(1-methylethyl)-5-methyl-3-pyrazolidinone (VII.1)

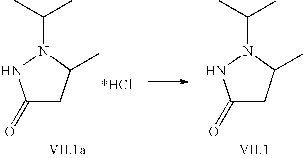

1-(1-Methylethyl)-5-methyl-3-pyrazolidinone monohydrochloride (150 g; 0.84 mol) is treated with saturated aqueous potassium carbonate (1.2 L) and ethyl acetate (1.0 L). The mixture is filtered and the phases are separated. The organic phase is dried with anhydrous sodium sulphate, filtered and evaporated in vacuo to yield 1-(1-methylethyl)-5-methyl-3-pyrazolidinone as a solid.

Mass and ¹H-NMR-spectra are in accordance with the assigned structure.

Mass spectrum (ESI⁺): m/z=143 [M+H]⁺

### EXAMPLE 1.4

### Preparation of 1,2-dihydro-11-methylethyl)-5-methyl-3H-pyrazol-3-one (VI.1)

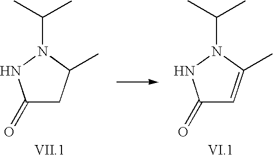

Variant 1:

1-(1-methylethyl)-5-methyl-3-pyrazolidinone (390 g; 2.74 mol) is dissolved in acetic acid (170 ml) with warming. 35% aqueous hydrogen peroxide (260 ml; 3.0 mol) is added within 3 h while keeping the temperature at about 65° C. The reaction mixture is then stirred at about 20 to 25° C. for 15 h. Water (1.2 L) is then added and the pH of the mixture is adjusted to about 7 by means of addition of approx. 1 L 50%-weight aqueous sodium hydroxide solution. Upon cooling to 5° C. the reaction mixture is filtered. The product is washed with water and dried at about 50° C. Colourless crystals are obtained.

Variant 2:

To a solution of 77 g potassium carbonate in 150 mL of water is added 50 g (279.86 mmol) 1-(1-methylethyl)-5-methyl-3-pyrazolidinone monohydrochloride (VII.1a) followed by 250 mL isopropyl acetate. The mixture is heated to 50° C. with stirring for 5 min. after which the aqueous phase is seperated. From the organic phase 175 mL of solvent is distilled off in vacuo. The remainder solution is filtered and the filter is washed with 50 mL isopropyl acetate. The combined organic phases are concentrated in vacuo to an oil. To the latter is added 50 mL acetic acid and the mixture is cooled to approx. 3° C. 66.9 g of peracetic acid is added together with 12.5 mL acetic acid. The mixture is stirred at 3° C. for approx. 1 h. 325 mL of water is then added and the pH of the solution is adjusted to 6.6-7.0 by means of addition of 50% aqueous sodium hydroxide. The resulting suspension is stirred for 30 min. at 10° C. after which it is filtered. The product is washed with water and dried at 45° C.

Mass and ¹H-NMR-spectra are in accordance with the assigned structure.

Mass spectrum (ESI⁺): m/z=141 [M+H]⁺

### EXAMPLE 2

### Synthesis of 1,2-dihydro-1-cyclobutyl-5-methyl-3H-pyrazol-3-one (VI.2)

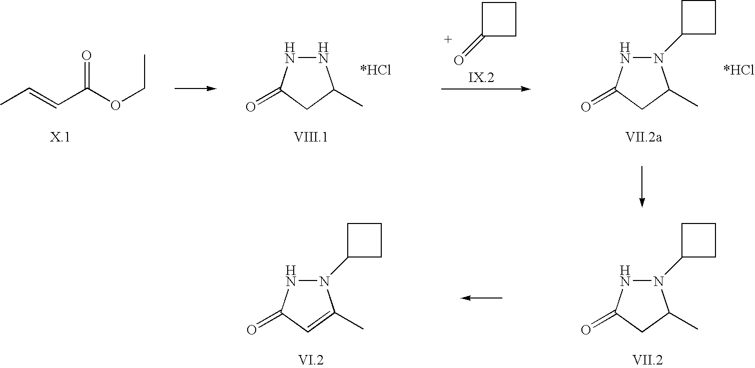

The synthesis of the intermediate VIII.1 is described in example 1.1. The intermediate VII.2a may be obtained by employing the example 1.2 wherein instead of propanon the appropriate amount of cyclobutanon (IX.2) is taken. Starting from the intermediate VII.2a the compounds VII.2 and VI.2 may be obtained by using the procedure as described in the examples 1.3 and 1.4 in an analogous manner.

### EXAMPLE 3a

### Synthesis of 1,2-Dihydro-1-(1-methylethyl)-4-[(2-fluor-4-methoxyphenyl)-(1-pyrrolidino)methyl]-5-methyl-3H-pyrazol-3-one (IV.1a)

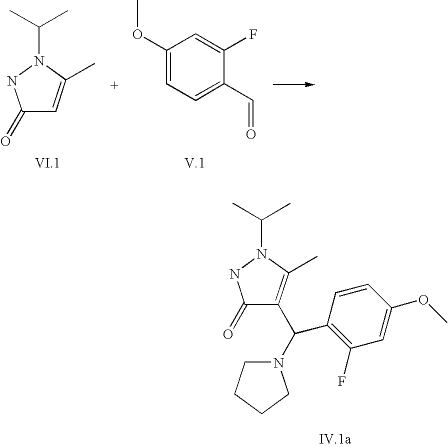

To a mixture of 70 g (0.50 mol) 1,2-dihydro-1-(1-methylethyl)-5-methyl-3H-pyrazol-3-one and 350 mL acetonitrile is added a solution of 77 g (0.50 mol) 2-fluor-4-methoxybenzaldehyd in 280 mL acetonitrile. Acetic acid 6 g and pyrrolidine 53.3 g (0.75 mol) are added sequentially at 20° C. to the reaction mixture together with 70 mL acetonitrile. The reaction mixture is heated to 75° C. for 1 h after which it is cooled to 3° C. The cooled reaction mixture is stirred for further 30 minutes after which the product is isolated by filtration. It is washed twice with 140 mL cold acetonitrile each and is subsequently dried under inert atmosphere at 40° C.

### EXAMPLE 3b

### Synthesis of 1,2-Dihydro-1-(1-methylethyl)-4-[(2-fluor-4-methoxyphenyl)-(ethoxy)methyl]-5-methyl-3H-pyrazol-3-one (IV.1b)

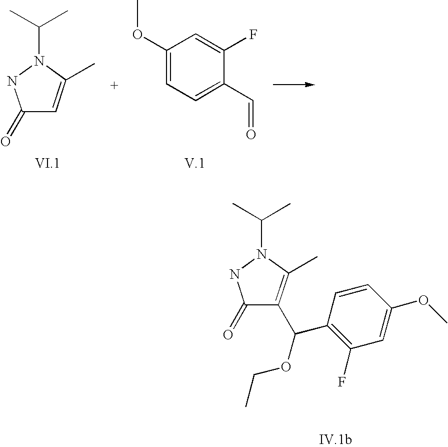

Variant 1:

Pyrrolidine (21 ml; 0.257 mol) and acetic acid (22 ml; 0.385 mol) are added to a mixture of 1,2-dihydro-1-(1-methylethyl)-5-methyl-3H-pyrazol-3-one (180 g; 1.28 mol) and 2-fluoro-4-methoxybenzaldehyde (198 g; 1.28 mol) in ethanol (2.7 L). The suspension is heated to about 50° C. for about 67 h. The reaction mixture is then cooled to approx. 17° C. and filtered. The product is washed with diisopropyl ether (500 ml) and subsequently refluxed with THF (2.5 L). The obtained solution is filtered over a pad of Celite and charcoal. The filtrate is concentrated in vacuo and water (2 L) is added to the suspension which is cooled and filtered. The colourless crystals are dried at 50° C.

Variant 2:

To a suspension of 1,2-dihydro-1-(1-methylethyl)-5-methyl-3H-pyrazol-3-one 7 g (50 mmol) in 50 ml ethanol is added 2-fluor-4-methoxybenzaldehyd 7.7 g (50 mmol) in 20 ml ethanol. Acetic acid 0.6 9 (10 mmol) is added followed by addition of pyrrolidine 5.33 g (75 mmol). The reaction mixture is then heated to 70° C. for 1-2 hrs after which it is cooled to approx. 20° C. Aqueous 30% hydrochloric acid (65 mmol) is then added and the reaction mixture is heated to 50° C. for 3 h. Water is then added and the mixture is cooled. The product is filtered and washed with 50% aqueous ethanol. It is dried at 45° C. under inert gas atmosphere.

Variant 3:

To a mixture of 50 g (0,144 mol) 1,2-dihydro-1-(1-methylethyl)-4-[(2-fluor-4-methoxyphenyl)-(1-pyrrolidino)methyl]-5-methyl-3H-pyrazol-3-one and 500 ml ethanol is added aq. 30% hydrochloric acid. The reaction mixture is heated to 50° C. for 2-3 h. 175 ml of water is then added and the mixture is cooled. The product is isolated by filtration and washed with ethanol. It is dried under inert gas atmosphere at 45° C.

Mass and ¹H-NMR-spectra are in accordance with the assigned structure.

Mass spectrum (ESI⁺): m/z=323 [M+H]⁺

### EXAMPLE 4

### Synthesis of 1,2-Dihydro-1-(1-methylethyl)-4-[(2,3-difluoro-4-methoxyphenyl)-(ethoxy)methyl]-5-methyl-3H-pyrazol-3-one (IV.2)

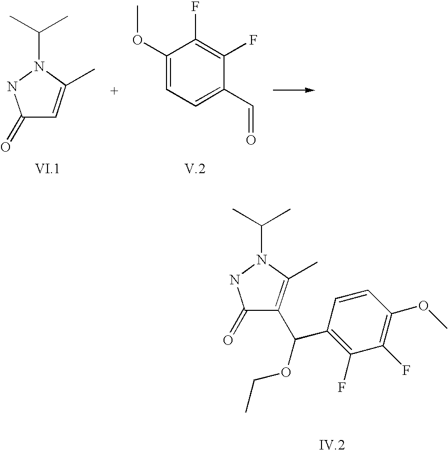

The intermediate of the formula IV.2 may be obtained using the procedure as described in the example 3b in an analogous manner.

### EXAMPLE 5

### Synthesis of 1,2-Dihydro-1-(1-methylethyl)-4-[(2,6-difluoro-4-methoxyphenyl)-(ethoxy)methyl]-5-methyl-3H-pyrazol-3-one (IV.3)

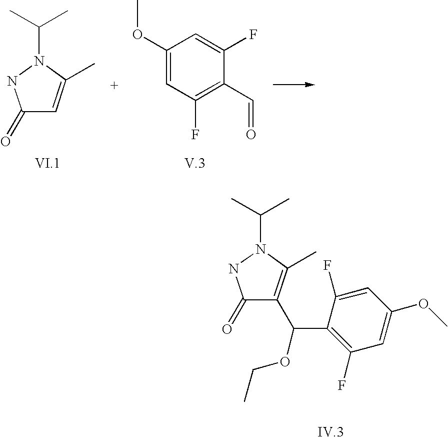

The intermediate of the formula IV.3 may be obtained using the procedure as described in the example 3b in an analogous manner.

### EXAMPLE 6

### Synthesis of 1,2-Dihydro-1-cyclobutyl-4-[(3-fluoro-4-methylphenyl)-(ethoxy)methyl]-5-methyl-3H-pyrazol-3-one (IV.4)

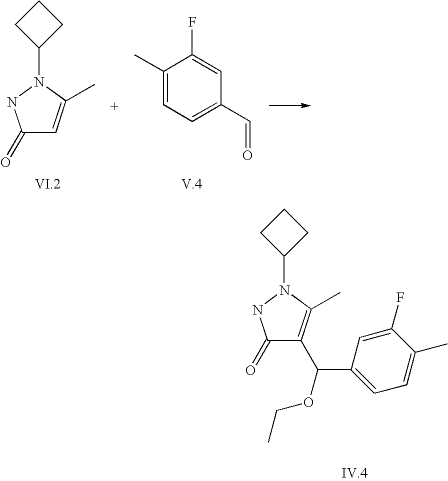

The intermediate of the formula IV.4 may be obtained using the procedure as described in the example 3b in an analogous manner.

### EXAMPLE 7

### Synthesis of 1,2-Dihydro-1-cyclobutyl-4-[(2-fluoro-4-methoxyphenyl)-(ethoxy)methyl]-5-methyl-3H-pyrazol-3-one (IV.5)

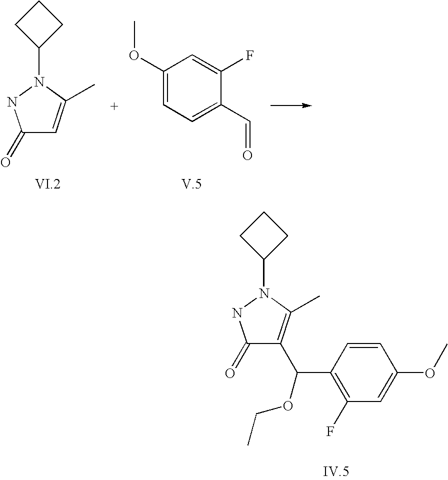

The intermediate of the formula IV.5 may be obtained using the procedure as described in the example 3b in an analogous manner.

### EXAMPLE 8

### Synthesis of 1,2-Dihydro-1-(1-methylethyl)-4-[(2,3-difluoro-4-methylphenyl)-(ethoxy)methyl]-5-methyl-3H-pyrazol-3-one (IV.6)

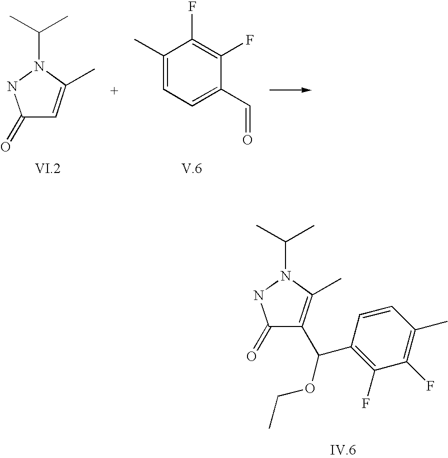

The intermediate of the formula IV.6 may be obtained using the procedure as described in the example 3b in an analogous manner.

### EXAMPLE 9

### Synthesis of 1,2-Dihydro-1-(1-methylethyl)-4-[(2-fluoro-4-methylphenyl)-(ethoxy)methyl]-5-methyl-3H-pyrazol-3-one (IV.7)

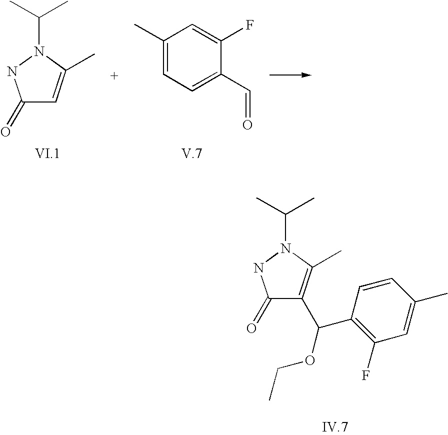

The intermediate of the formula IV.7 may be obtained using the procedure as described in the example 3b in an analogous manner.

### EXAMPLE 10

### Synthesis of 1,2-Dihydro-1-(1-methylethyl)-4-[(3-fluoro-4-ethoxyphenyl)-(ethoxy)methyl]-5-methyl-3H-pyrazol-3-one (IV.8)

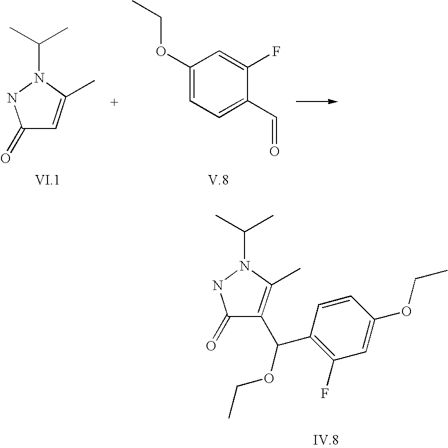

The intermediate of the formula IV.8 may be obtained using the procedure as described in the example 3b in an analogous manner.

### EXAMPLE 11

### Synthesis of 1,2-Dihydro-1-(1-methylethyl)-4-[(3-fluoro-4-(1-methylethoxy)phenyl)-(ethoxy)methyl]-5-methyl-3H-pyrazol-3-one (IV.9)

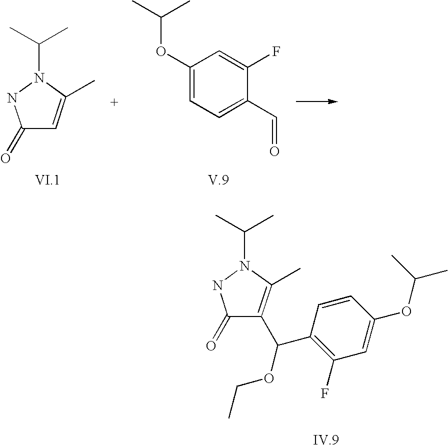

The intermediate of the formula IV.9 may be obtained using the procedure as described in the example 3b in an analogous manner.

### EXAMPLE 12

### Synthesis of 1,2-Dihydro-1-(1-methylethyl)-4-[(2-fluoro-4-(1-methylethoxy)phenyl)-(ethoxy)methyl]-5-methyl-3H-pyrazol-3-one (IV.10)

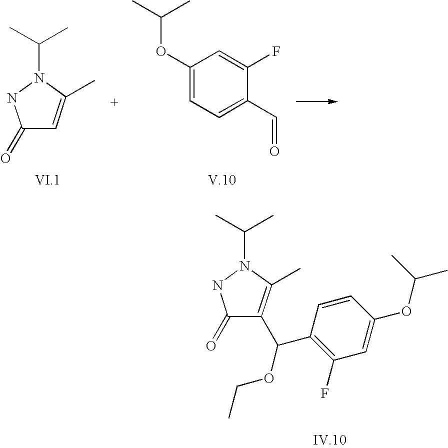

The intermediate of the formula IV.10 may be obtained using the procedure as described in the example 3b in an analogous manner.

### EXAMPLE 13

### Synthesis of 1,2-Dihydro-1-(1-methylethyl)-4-[(2-fluoro-4-ethoxyphenyl)-(ethoxy)methyl]-5-methyl-3H-pyrazol-3-one (IV.11)

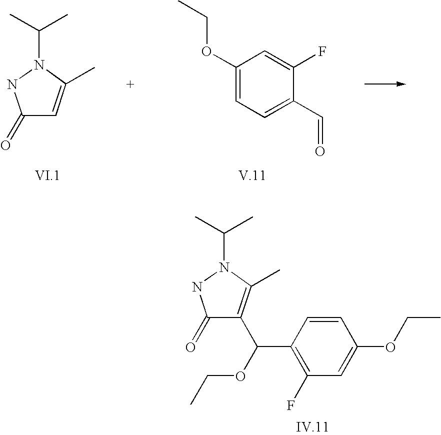

The intermediate of the formula IV.11 may be obtained using the procedure as described in the example 3b in an analogous manner.

### EXAMPLE 14

### Synthesis of 1,2-Dihydro-1-(1-methylethyl)-4-[(2-fluor-4-methoxyphenyl)methyl]-5-methyl-3H-pyrazol-3-one (III.1)

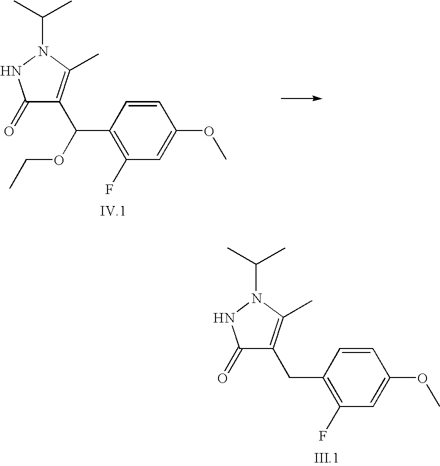

A mixture of 1,2-dihydro-1-(1-methylethyl)-4-[(2-fluor-4-methoxyphenyl)-(ethoxy)methyl]-5-methyl-3H-pyrazol-3-one (294 g; 1.28 mol), methanol (4.5 L), aqueous hydrochloric acid (30%; 11 g) and water (80 mL) is hydrogenated with palladium on charcoal (10%-weight) (65 g) at 50° C. and 3 bar hydrogen pressure until hydrogen uptake ceases. THF (2.3 L) is added to the reaction mixture which is then filtered. The catalyst is washed with THF (1 L) and the solvent is distilled off under reduced pressure to a residual volume of approx. 700 to 800 ml. The resulting suspension is poured into water (1 L) with stirring. The precipitate is isolated by filtration, washed with water (400 ml) and dried at 55° C. to yield crystals (light beige).

Mass and ¹H-NMR-spectra are in accordance with the assigned structure.

Mass spectrum (ESI⁺): m/z=279 [M+H]⁺

### EXAMPLE 15

### Synthesis of 1,2-Dihydro-1-(1-methylethyl)-4-[(2,3-difluoro-4-methoxyphenyl)methyl]-5-methyl-3H-pyrazol-3-one (III.2)

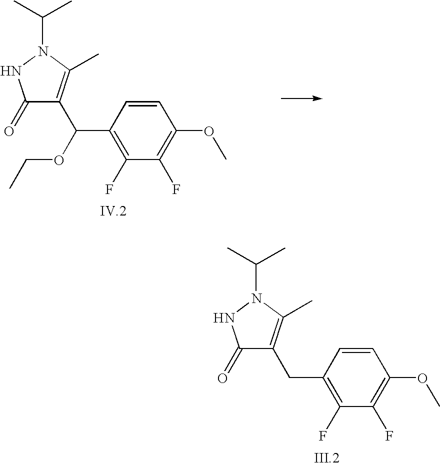

The compound of the formula III.2 may be obtained by employing the synthetic procedure as outlined in example 14 in an analogous manner.

### EXAMPLE 16

### Synthesis of 1,2-Dihydro-1-(1-methylethyl)-4-[(2,6-difluoro-4-methoxyphenyl)methyl]-5-methyl-3H-pyrazol-3-one (III.3)

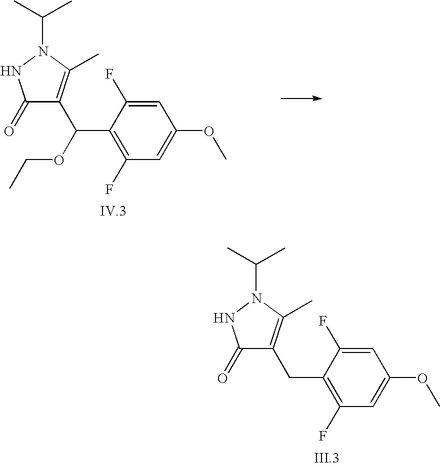

The compound of the formula III.3 may be obtained by employing the synthetic procedure as outlined in example 14 in an analogous manner.

### EXAMPLE 17

### Synthesis of 1,2-Dihydro-1-(1-cyclobutyl)-4-[(3-fluoro-4-methylphenyl)methyl]-5-methyl-3H-pyrazol-3-one (III.4)

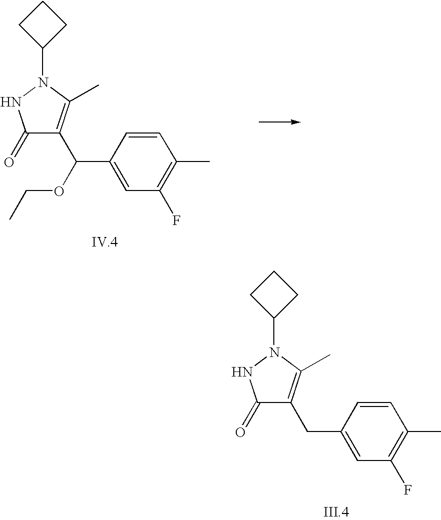

The compound of the formula III.4 may be obtained by employing the synthetic procedure as outlined in example 14 in an analogous manner.

### EXAMPLE 18

### Synthesis of 1,2-Dihydro-1-(1-cyclobutyl)-4-[(2-fluoro-4-methoxyphenyl)methyl]-5-methyl-3H-pyrazol-3-one (III.5)

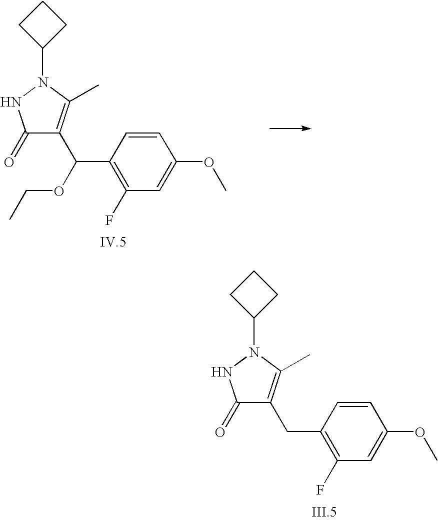

The compound of the formula III.5 may be obtained by employing the synthetic procedure as outlined in example 14 in an analogous manner.

### EXAMPLE 19

### Synthesis of 1,2-Dihydro-1-(1-methylethyl)-4-[(2,3-difluoro-4-methylphenyl)methyl]-5-methyl-3H-pyrazol-3-one (III.6)

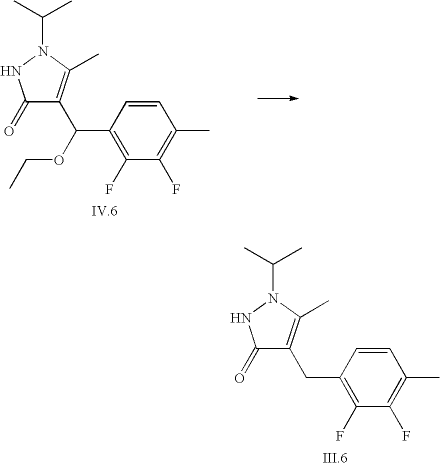

The compound of the formula III.6 may be obtained by employing the synthetic procedure as outlined in example 14 in an analogous manner.

### EXAMPLE 20

### Synthesis of 1,2-Dihydro-1-(1-methylethyl)-4-[(2-fluoro-4-methylphenyl)methyl]-5-methyl-3H-pyrazol-3-one (III.7)

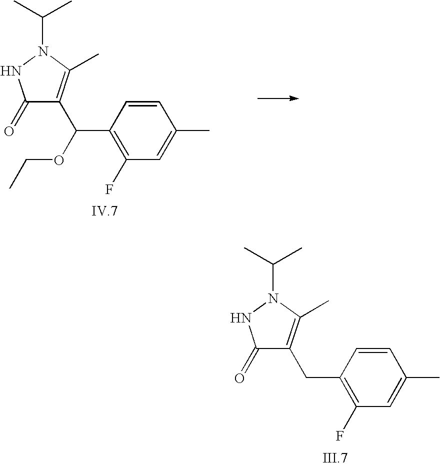

The compound of the formula 111.7 may be obtained by employing the synthetic procedure as outlined in example 14 in an analogous manner.

### EXAMPLE 21

### Synthesis of 1,2-Dihydro-1-(1-methylethyl)-4-[(3-fluoro-4-ethoxyphenyl)methyl]-5-methyl-3H-pyrazol-3-one (III.8)

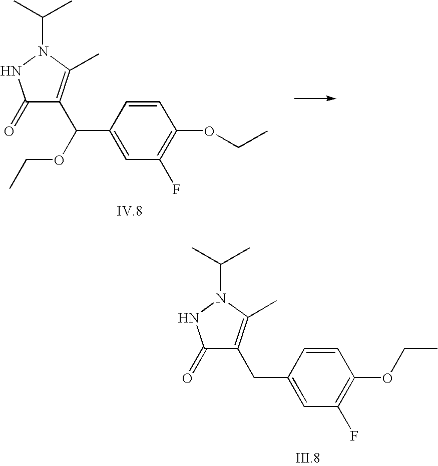

The compound of the formula III.8 may be obtained by employing the synthetic procedure as outlined in example 14 in an analogous manner.

### EXAMPLE 22

### Synthesis of 1,2-Dihydro-1-(1-methylethyl)-4-[(3-fluoro-4-(1-methylethoxy)-phenyl)methyl]-5-methyl-3H-pyrazol-3-one (III.9)

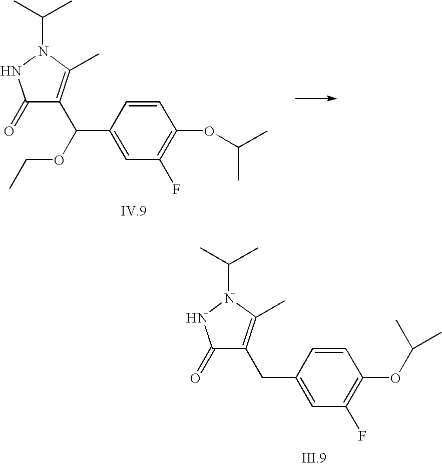

The compound of the formula III.9 may be obtained by employing the synthetic procedure as outlined in example 14 in an analogous manner.

### EXAMPLE 23

### Synthesis of 1,2-Dihydro-1-(1-methylethyl)-4-[(2-fluoro-4-(1-methylethoxy)-phenyl)methyl]-5-methyl-3H-pyrazol-3-one (III.10)

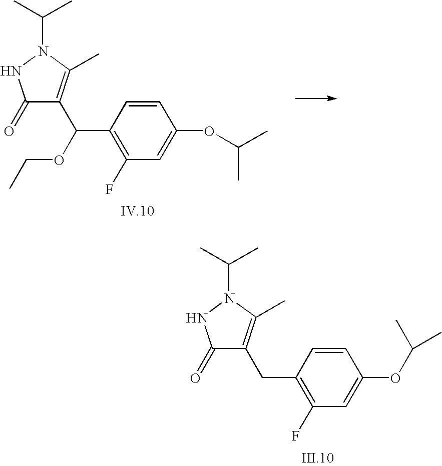

The compound of the formula III.10 may be obtained by employing the synthetic procedure as outlined in example 14 in an analogous manner.

### EXAMPLE 24

### Synthesis of 1,2-Dihydro-1-(1-methylethyl)-4-[(2-fluoro-4-ethoxyphenyl)methyl]-5-methyl-3H-pyrazol-3-one (III.11)

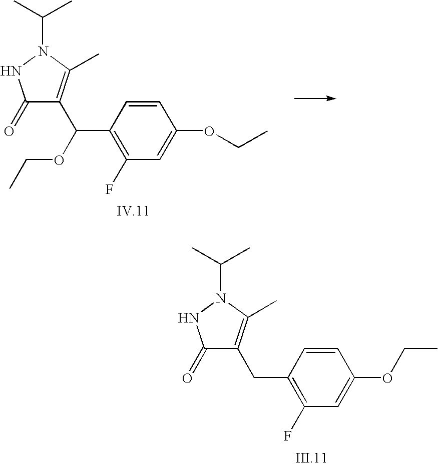

The compound of the formula III.11 may be obtained by employing the synthetic procedure as outlined in example 14 in an analogous manner.

### EXAMPLE 25

### Synthesis of 2,3,4,6-Tetra-O-acetyl-α-D-glucopyranosyl bromide (II.1)

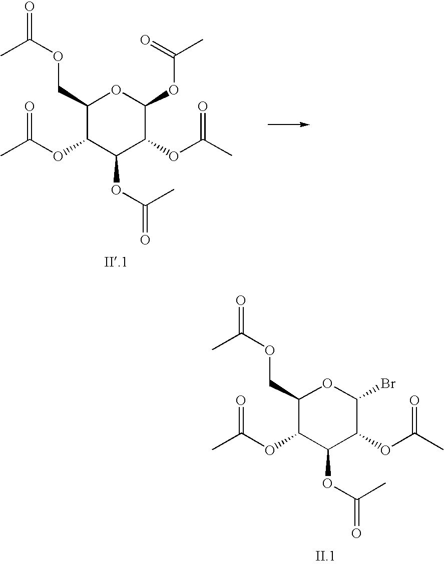

1,2,3,4,6-Penta-O-acetyl-α-D-glucopyranose (100 g, 0,251 mol) is suspended in toluene (210 ml). Acetic anhydride (9,5 ml; 0,1 mol) is added followed by hydrobromic acid 30% in acetic acid (200 ml, 1 mol). The mixture is stirred at 18° C. for 30 min. A mixture of ice/water (300 ml) and brine (100 ml) is then added with stirring. The phases are separated and the aqueous phase is extracted with toluene (100 ml). The organic phases are combined and washed with aqueous sodium hydrogencarbonate (100 ml) and brine (100 ml). Dyring and evaporation of the solvent under reduced pressure yields an oil which is crystallised by addition of methyl-t-butylether (150 ml) and methylcyclohexane (300 ml). The product is isolated by filtration, washed with methylcyclohexane and dried under vacuo at 45° C.

### EXAMPLE 26

### Synthesis of 1′-(1-methylethyl)-4′-[(2-fluoro-4-methoxyphenyl)methyl]-5′-methyl-1H-pyrazol-3′-O-(2,3,4,6-O-tetraacetyl)-β-D-glucopyranoside (I.1)

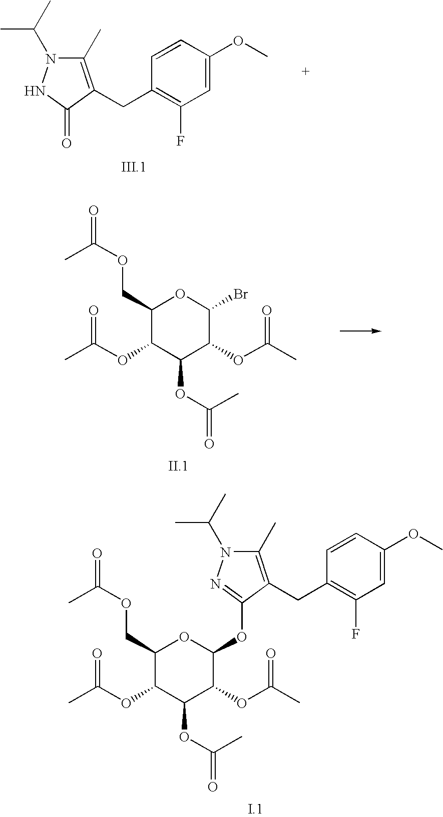

Aqueous potassium hydroxide (1M; 870 ml) is added to a mixture of (2,3,4,6-O-tetraacetyl)-α-D-glucopyranosyl bromide (485 g; 1.169 mol), 1,2-dihydro-1-(1-methylethyl)-4-[(2-fluor-4-methoxyphenyl)methyl]-5-methyl-3H-pyrazol-3-one (161 g; 0.58 mol) and tetrabutylammonium chloride (9.4 g; 0.029 mol ) in dichloromethane (780 L). The two phase mixture is vigorously stirred at 25 to 27° C. while the pH of the aqueous layer is kept constant at approx. 13 by adding further aqueous potassium hydroxide (4N; about 870 ml ) until consumption of base ceases (about 5 h). The progress of the reaction can be monitored by HPLC. The phases are then separated and the aqueous phase is extracted with dichloromethane (800 ml). The combined organic phases are dried (Na₂SO₄), filtered and evaporated under reduced pressure to yield an oil (yellow). This is used in the next reaction step without further purification.

Mass and ¹H-NMR-spectra are in accordance with the assigned structure.

Mass spectrum (ESI⁺): m/z=609 [M+H]⁺

### EXAMPLE 27

### Synthesis of 1′-(1-methylethyl)-4′-[(2,3-difluoro-4-methoxyphenyl)methyl]-5′-methyl-1H-pyrazol-3′-O-(2,3,4,6-O-tetraacetyl)-β-D-glucopyranoside (I.2)

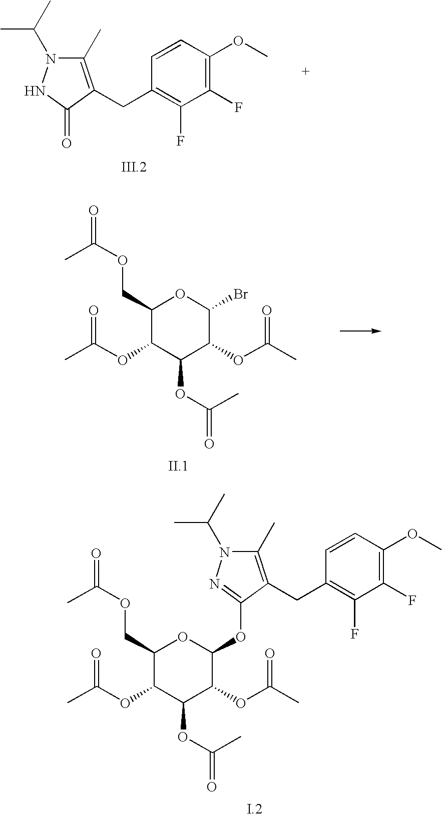

The compound of the formula I.2 may be obtained by employing the synthetic procedure as outlined in example 26 in an analogous manner.

### EXAMPLE 28

### Synthesis of 1′-(1-methylethyl)-4′-[(2,6-difluoro-4-methoxyphenyl)methyl]-5′-methyl-1H-pyrazol-3′-O-(2,3,4,6-O-tetraacetyl)-β-D-glucopyranoside (I.3)

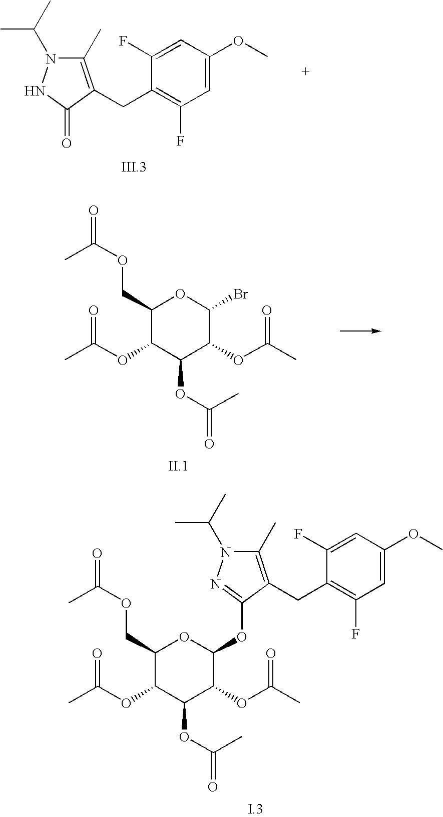

The compound of the formula I.3 may be obtained by employing the synthetic procedure as outlined in example 26 in an analogous manner.

### EXAMPLE 29

### Synthesis of 1′-(1-cyclobutyl)-4′-[(3-fluoro-4-methylphenyl)methyl]-5′-methyl-1H-pyrazol-3′-O-(2,3,4,6-O-tetraacetyl)-β-D-glucopyranoside (I.4)

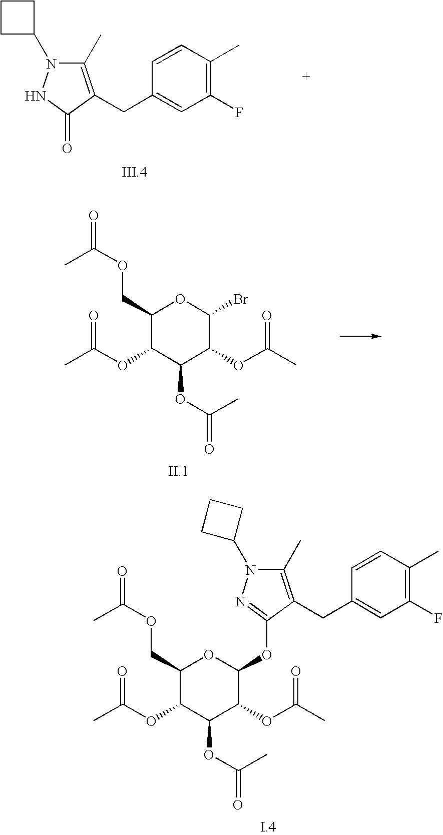

The compound of the formula I.4 may be obtained by employing the synthetic procedure as outlined in example 26 in an analogous manner.

### EXAMPLE 30

### Synthesis of 1′-(1-cyclobutyl)-4′-[(2-fluoro-4-methoxyphenyl)methyl]-5′-methyl-1H-pyrazol-3′-O-(2,3,4,6-O-tetraacetyl)-β-D-glucopyranoside (I.5)

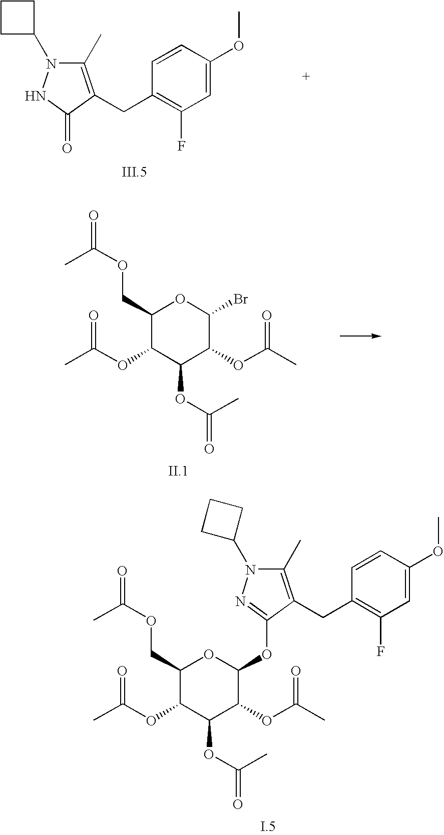

The compound of the formula I.5 may be obtained by employing the synthetic procedure as outlined in example 26 in an analogous manner.

### EXAMPLE 31

### Synthesis of 1′-(1-methylethyl)-4′-[(2,3-difluoro-4-methylphenyl)methyl]-5′-methyl-1H-pyrazol-3′-O-(2,3,4,6-O-tetraacetyl)-β-D-glucopyranoside (I.6)

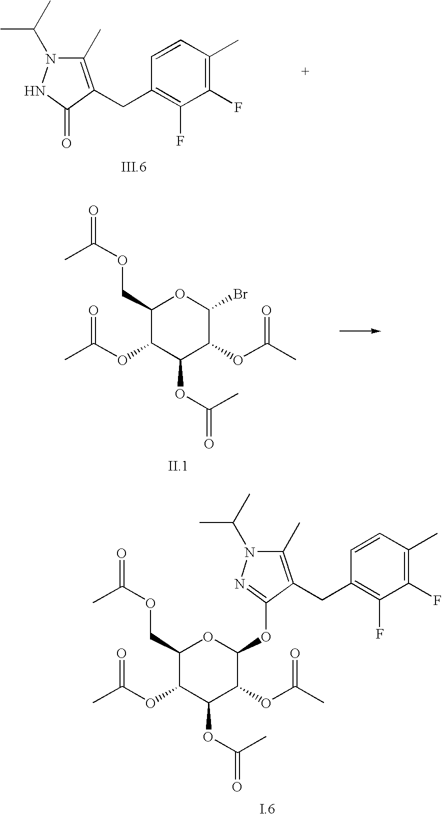

The compound of the formula I.6 may be obtained by employing the synthetic procedure as outlined in example 26 in an analogous manner.

### EXAMPLE 32

### Synthesis of 1′-(1-methylethyl)-4′-[(2-fluoro-4-methylphenyl)methyl]-5′-methyl-1H-pyrazol-3′-O-(2,3,4,6-O-tetraacetyl)-β-D-glucopyranoside (I.7)

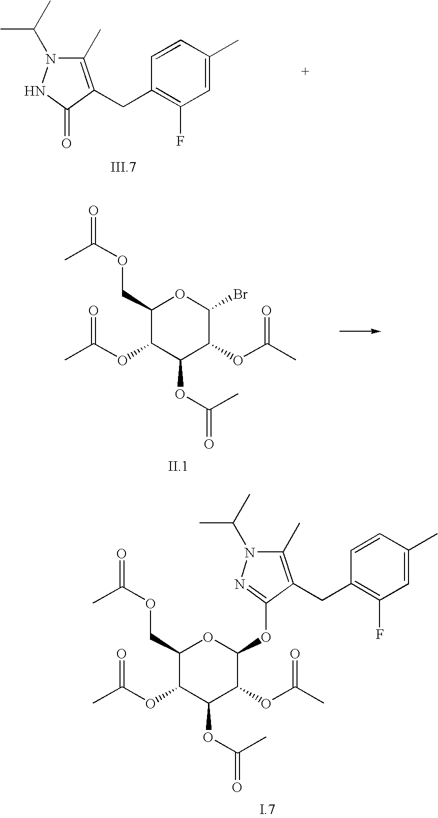

The compound of the formula I.7 may be obtained by employing the synthetic procedure as outlined in example 26 in an analogous manner.

### EXAMPLE 33

### Synthesis of 1′-(1-methylethyl)-4′-[(3-fluoro-4-ethoxyphenyl)methyl]-5′-methyl-1H-pyrazol-3′-O-(2,3,4,6-O-tetraacetyl)-β-D-glucopyranoside (I.8)

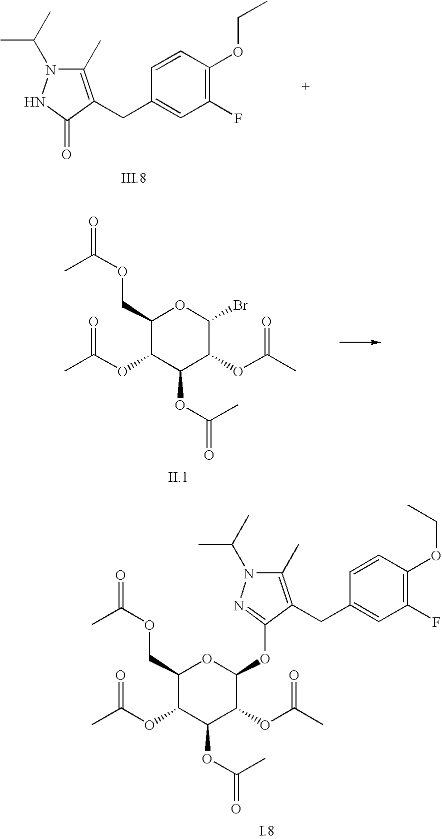

The compound of the formula I.8 may be obtained by employing the synthetic procedure as outlined in example 26 in an analogous manner.

### EXAMPLE 34

### Synthesis of 1′-(1-methylethyl)-4′-[(3-fluoro-4-(1-methylethoxy)phenyl)methyl]-5′-methyl-1H-pyrazol-3′-O-(2,3,4,6-O-tetraacetyl)-β-D-glucopyranoside (I.9)

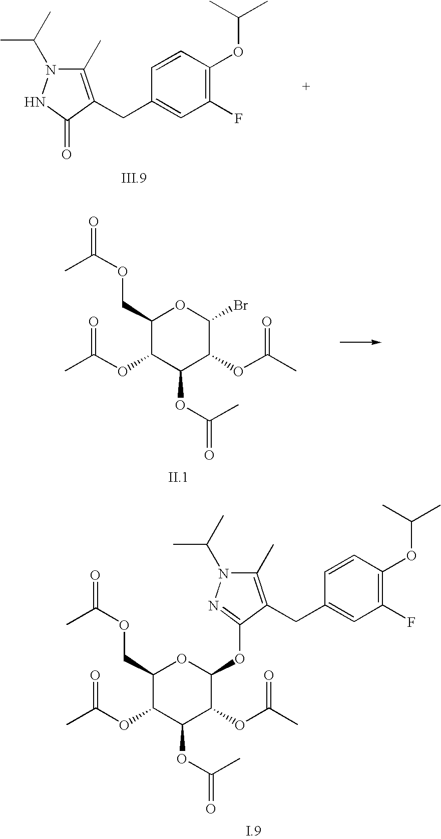

The compound of the formula 1.9 may be obtained by employing the synthetic procedure as outlined in example 26 in an analogous manner.

### EXAMPLE 35

### Synthesis of 1′-(1-methylethyl)-4′-[(2-fluoro-4-(1-methylethoxy)phenyl)methyl]-5′-methyl-1H-pyrazol-3′-O-(2,3,4,6-O-tetraacetyl)-β-D-glucopyranoside (I.10)

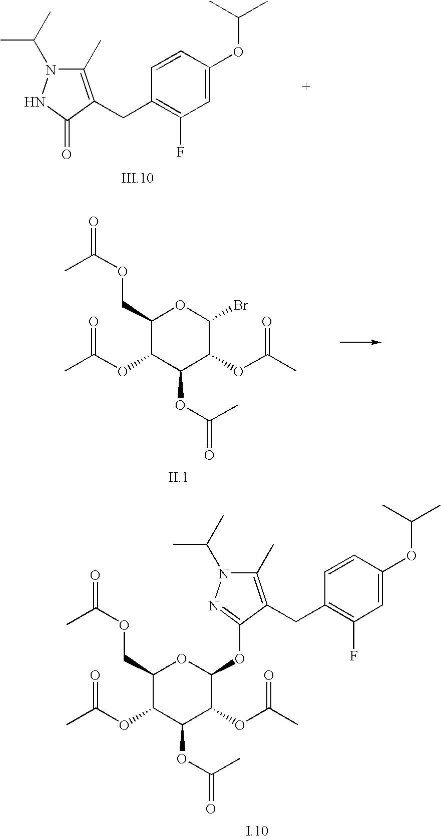

The compound of the formula I.10 may be obtained by employing the synthetic procedure as outlined in example 26 in an analogous manner.

### EXAMPLE 36

### Synthesis of 1′-(1-methylethyl)-4′-[(2-fluoro-4-ethoxyphenyl)methyl]-5′-methyl-1H-pyrazol-3′-O-(2,3,4,6-O-tetraacetyl)-β-D-glucopyranoside (I.11)

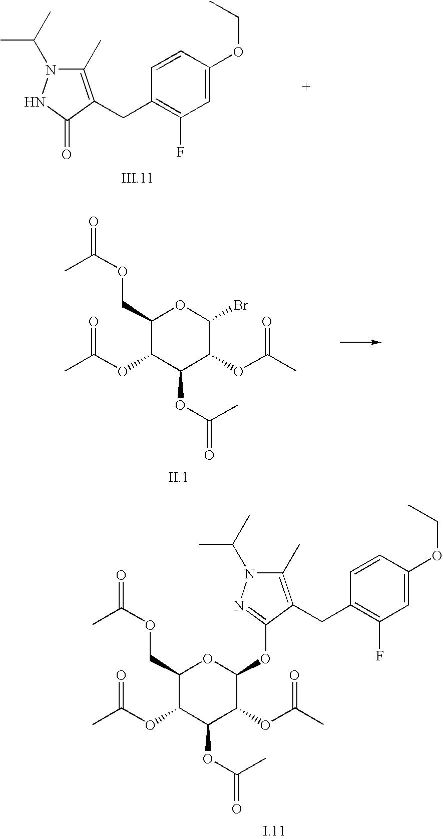

The compound of the formula I.11 may be obtained by employing the synthetic procedure as outlined in example 26 in an analogous manner.

### EXAMPLE 37

### Synthesis of 1′-(1-methylethyl)-4′-[(2-fluoro-4-methoxyphenyl)methyl]-5′-methyl-1H-pyrazol-3′-O-β-D-glucopyranoside (IH.1)

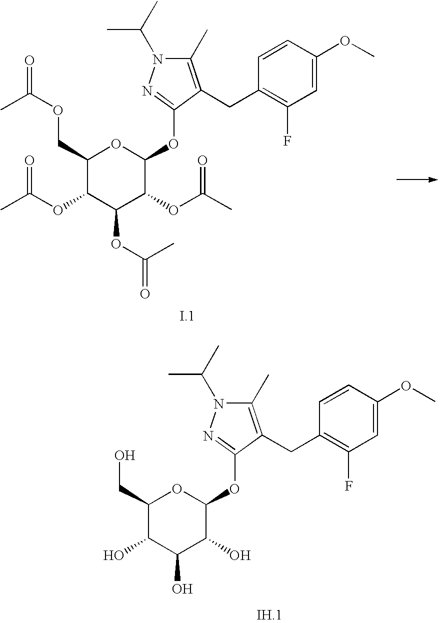

Crude 1′-(1-methylethyl)-4′-[(2-fluoro-4-methoxyphenyl)methyl]-5′-methyl-1H-pyrazol-3′-O-(2,3,4,6-O-tetraacetyl)-β-D-glucopyranoside (413 g; approx. 0.58 mol ) of the reaction according to example 26 is dissolved in dry ethanol (1 L ). Potassium-t-butoxide (6.6 g; 0.058 mol) is then added and the reaction mixture is stirred at about 20 to 25° C. for approx. 15 h. Acetic acid (3.3 ml; 0.058 mol) is then added and the solvent is distilled off under reduced pressure. The resulting residue is then dissolved in ethyl acetate (2 L) and washed with brine (800 ml). The organic phase is separated, dried over Na₂SO₄, filtered and evaporated under reduced pressure to yield a resinous solid.

Mass and ¹H-NMR-spectra are in accordance with the assigned structure.

Mass spectrum (ESI⁺): m/z=441 [M+H]⁺

### EXAMPLE 38

### Synthesis of 1′-(1-methylethyl)-4′-[(2,3-difluoro-4-methoxyphenyl)methyl]-5′-methyl-1H-pyrazol-3′-O-β-D-glucopyranoside (IH.2)

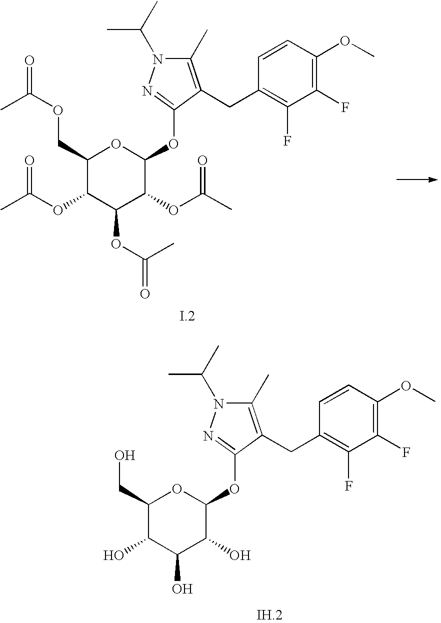

The compound of the formula IH.2 may be obtained by employing the synthetic procedure as outlined in example 37 in an analogous manner.

### EXAMPLE 39

### Synthesis of 1′-(1-methylethyl)-4′-[(2,6-difluoro-4-methoxyphenyl)methyl]-5′-methyl-1H-pyrazol-3′-O-β-D-glucopyranoside (IH.3)

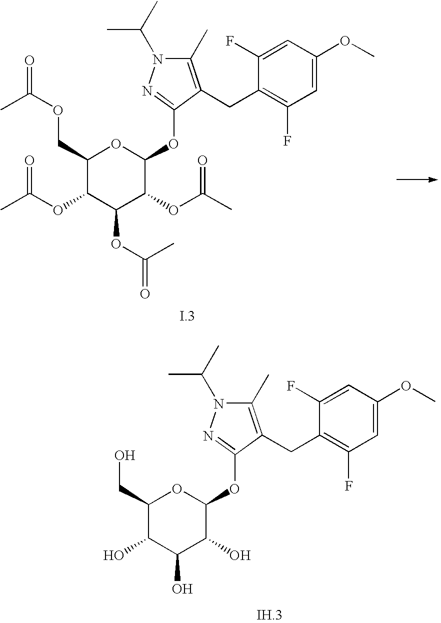

The compound of the formula IH.3 may be obtained by employing the synthetic procedure as outlined in example 37 in an analogous manner.

### EXAMPLE 40

### Synthesis of 1′-cyclobutyl-4′-[(3-fluoro-4-methylphenyl)methyl]-5′-methyl-1H-pyrazol-3′-O-β-D-glucopyranoside (IH.4)

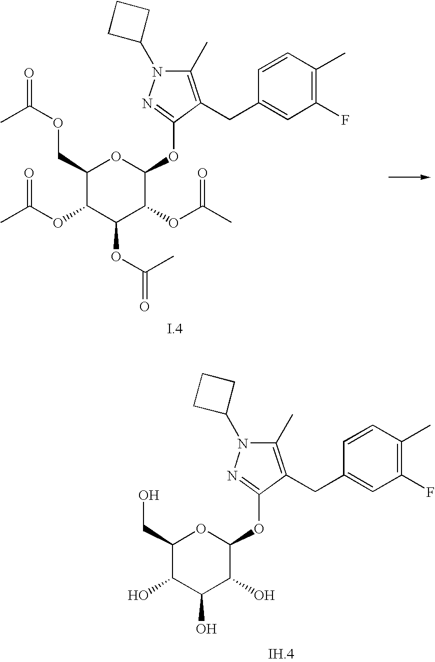

The compound of the formula IH.4 may be obtained by employing the synthetic procedure as outlined in example 37 in an analogous manner.

### EXAMPLE 41

### Synthesis of 1′-cyclobutyl-4′-[(2-fluoro-4-methoxyphenyl)methyl]-5′-methyl-1H-pyrazol-3′-O-β-D-glucopyranoside (IH.5)

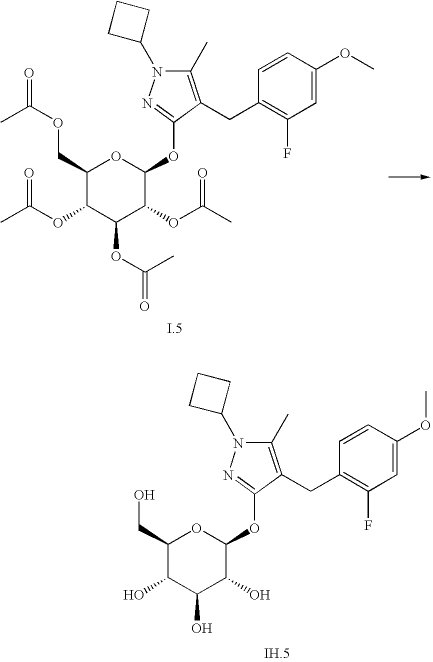

The compound of the formula IH.5 may be obtained by employing the synthetic procedure as outlined in example 37 in an analogous manner.

### EXAMPLE 42

### Synthesis of 1′-(1-methylethyl)-4′-[(2,3-difluoro-4-methylphenyl)methyl]-5′-methyl-1H-pyrazol-3′-O-β-D-glucopyranoside (IH.6)

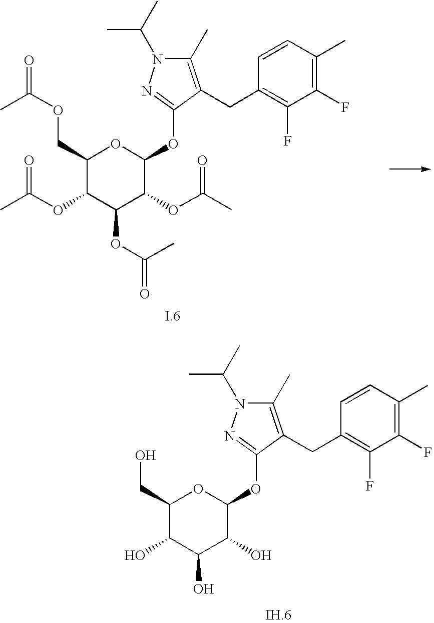

The compound of the formula IH.6 may be obtained by employing the synthetic procedure as outlined in example 37 in an analogous manner.

### EXAMPLE 43

### Synthesis of 1′-(1-methylethyl)-4′-[(2-fluoro-4-methylphenyl)methyl]-5′-methyl-1H-pyrazol-3′-O-β-D-glucopyranoside (IH.7)

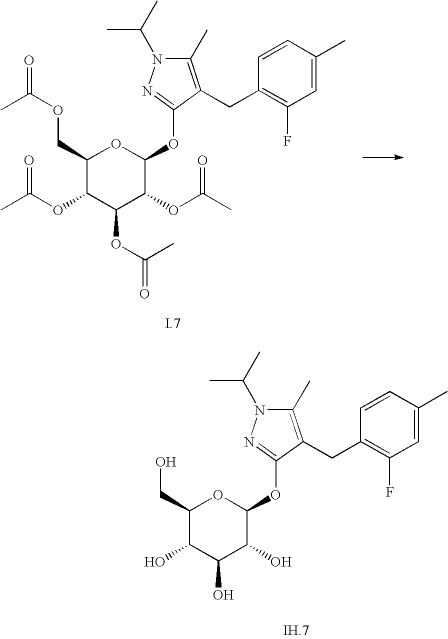

The compound of the formula IH.7 may be obtained by employing the synthetic procedure as outlined in example 37 in an analogous manner.

### EXAMPLE 44

### Synthesis of 1′-(1-methylethyl)-4′-[(3-fluoro-4-ethoxyphenyl)methyl]-5′-methyl-1H-pyrazol-3′-O-β-D-glucopyranoside (IH.8)

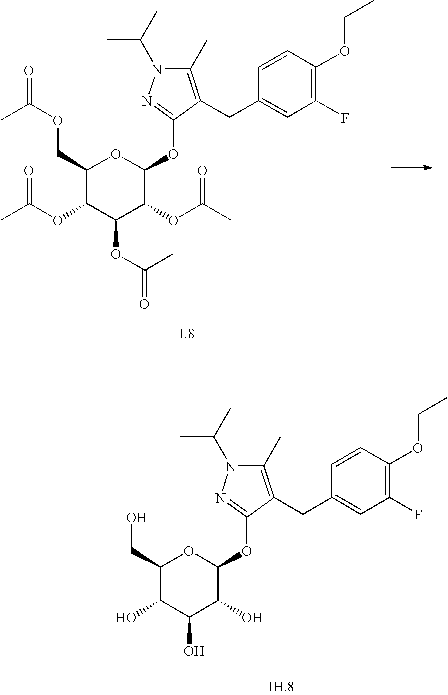

The compound of the formula IH.8 may be obtained by employing the synthetic procedure as outlined in example 37 in an analogous manner.

### EXAMPLE 45

### Synthesis of 1′-(1-methylethyl)-4′-[(3-fluoro-4-(1-methylethoxy)phenyl)methyl]-5′-methyl-1H-pyrazol-3′-O-β-D-glucopyranoside (IH.9)

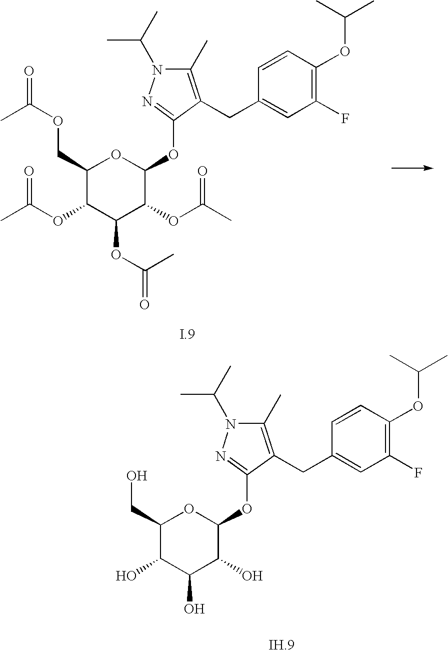

The compound of the formula IH.9 may be obtained by employing the synthetic procedure as outlined in example 37 in an analogous manner.

### EXAMPLE 46

### Synthesis of 1′-(1-methylethyl)-4′-[(2-fluoro-4-(1-methylethoxy)phenyl)methyl]-5′-methyl-1H-pyrazol-3′-O-β-D-glucopyranoside (IH.10)

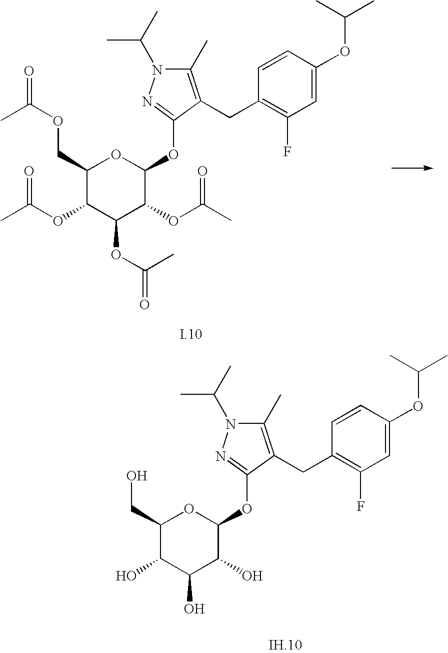

The compound of the formula IH.10 may be obtained by employing the synthetic procedure as outlined in example 37 in an analogous manner.

### EXAMPLE 47

### Synthesis of 1′-1-methylethyl)-4′-[(2-fluoro-4-ethoxyphenyl)methyl]-5-methyl-1H-pyrazol-3′-O-β-D-glucopyranoside (IH.11)

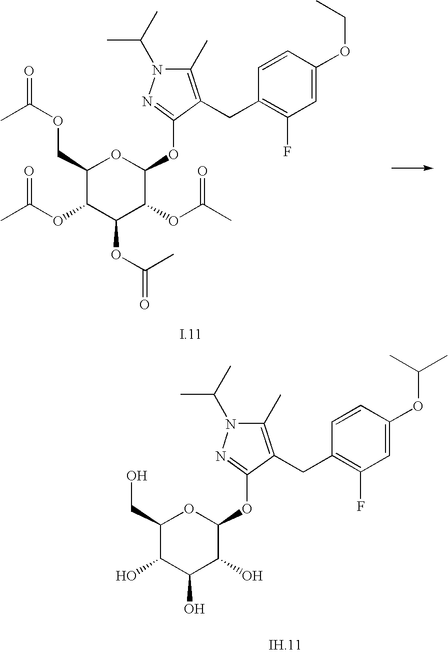

The compound of the formula IH.11 may be obtained by employing the synthetic procedure as outlined in example 37 in an analogous manner.

Examples on Alternative Synthesis of the Aglycon

### EXAMPLE 48

### Synthesis of 1,2-dihydro 4-[(2-fluor-4-methoxyphenyl)methyl]-5-methyl-3H-pyrazol-3-one (XI.1)

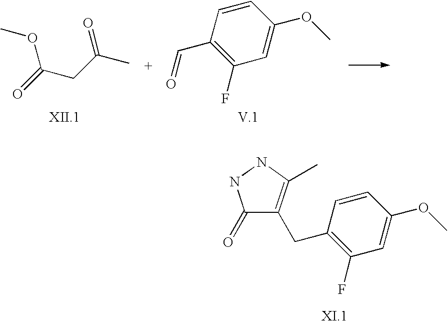

Piperidine (19.3 ml; 0.195 mol) is added to a mixture of methyl acetoacetate (71 mL; 0.65 mol), 2-fluoro-4-methoxy benzaldehyde (100 g; 0.65 mol) and acetic acid (37 ml; 0.65 mol) and the reaction mixture is stirred for 24 hrs at about 20 to 25° C. Isopropanol (500 ml) together with palladium (10%-weight) on charcoal (5 g) are then added and the solution is hydrogenated until uptake of hydrogen ceases. After removal of the catalyst by filtration hydrazine hydrate (43 mL; 0.7 mol) is added to the filtrate with stirring. The reaction mixture is heated to reflux for 3 hrs after which it is allowed to cool to about 20 to 25° C. The resulting suspension is then filtered, the product being washed with methyl-t-butyl ether and dried at 50° C. to yield colourless crystals.

Mass and ¹H-NMR-spectra are in accordance with the assigned structure. Mass spectrum (ESI⁺): m/z=237 [M+H]⁺

### EXAMPLE 49

### Synthesis of 1,2-dihydro 4-[(2,3-difluoro-4-methoxyphenyl)methyl]-5-methyl-3H-pyrazol-3-one (XI.2)

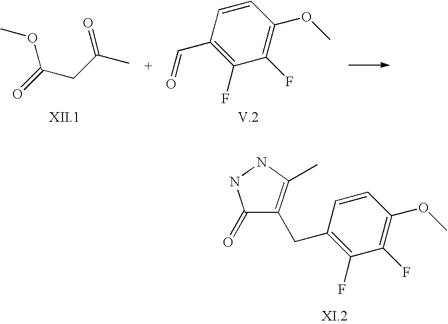

The compound of the formula XI.2 may be obtained by employing the synthetic procedure as outlined in example 48 in an analogous manner.

### EXAMPLE 50

### Synthesis of 1,2-dihydro 4-[(2,6-difluoro-4-methoxyphenyl)methyl]-5-methyl-3H-pyrazol-3-one (XI.3)

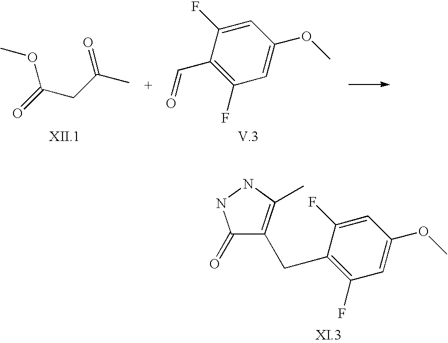

The compound of the formula XI.3 may be obtained by employing the synthetic procedure as outlined in example 48 in an analogous manner.

### EXAMPLE 51

### Synthesis of 1,2-dihydro 4-[(3-fluoro-4-methyl phenyl)methyl]-5-methyl-3H-pyrazol-3-one (XI.4)

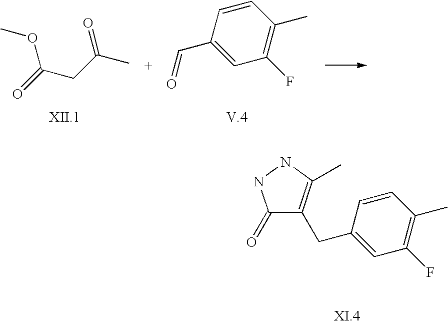

The compound of the formula XI.4 may be obtained by employing the synthetic procedure as outlined in example 48 in an analogous manner.

### EXAMPLE 52

### Synthesis of 1,2-dihydro 4-[(2,3-difluoro-4-methylphenyl)methyl]-5-methyl-3H-pyrazol-3-one (XI.6)

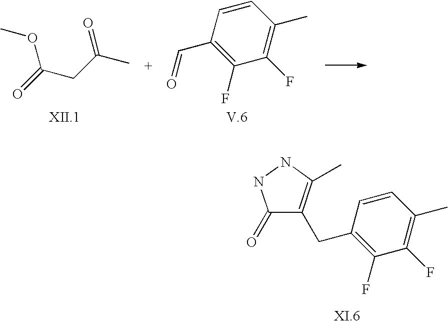

The compound of the formula XI.6 may be obtained by employing the synthetic procedure as outlined in example 48 in an analogous manner.

### EXAMPLE 53

### Synthesis of 1,2-dihydro 4-[(2-fluoro-4-methylphenyl)methyl]-5-methyl-3H-pyrazol-3-one (XI.7)

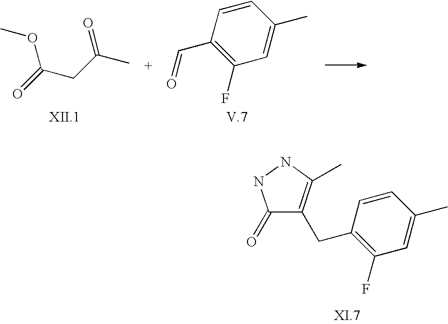

The compound of the formula XI.7 may be obtained by employing the synthetic procedure as outlined in example 48 in an analogous manner.

### EXAMPLE 54

### Synthesis of 1,2-dihydro 4-[(3-fluoro-4-ethoxyphenyl)methyl]-5-methyl-3H-pyrazol-3-one (XI.8)

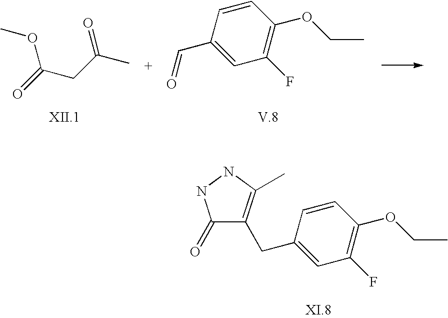

The compound of the formula XI.8 may be obtained by employing the synthetic procedure as outlined in example 48 in an analogous manner.

### EXAMPLE 55

### Synthesis of 1,2-dihydro 4-[(3-fluoro-4-(1-methylethoxy)phenyl)methyl]-5-methyl-3H-pyrazol-3-one (XI.9)

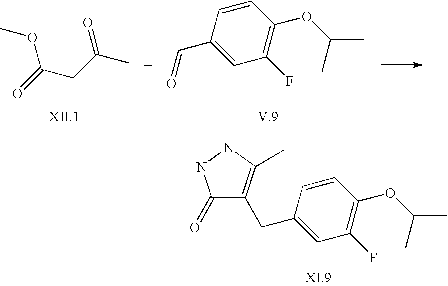

The compound of the formula XI.9 may be obtained by employing the synthetic procedure as outlined in example 48 in an analogous manner.

### EXAMPLE 56

### Synthesis of 1,2-dihydro 4-[(2-fluoro-4-(1-methylethoxy)phenyl)methyl]-5-methyl-3H-pyrazol-3-one (XI.10)

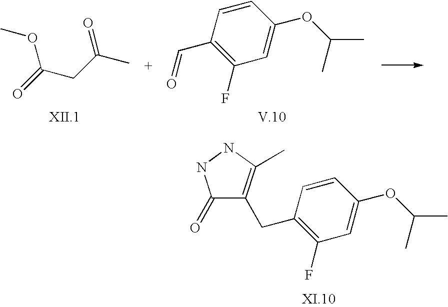

The compound of the formula XI.10 may be obtained by employing the synthetic procedure as outlined in example 48 in an analogous manner.

### EXAMPLE 57

### Synthesis of 1,2-dihydro 4-[(2-fluoro-4-ethoxyphenyl)methyl]-5-methyl-3H-pyrazol-3-one (XI.11)

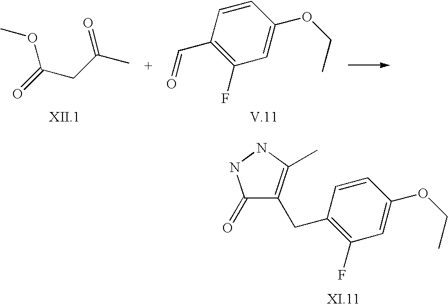

The compound of the formula XI.11 may be obtained by employing the synthetic procedure as outlined in example 48 in an analogous manner.

### EXAMPLE 58:

### 1,2-Dihydro-1-(1-methylethyl)-4-[(2-fluor-4-methoxyphenyl)methyl]-5-methyl-3H-pyrazol-3-one (III.1)

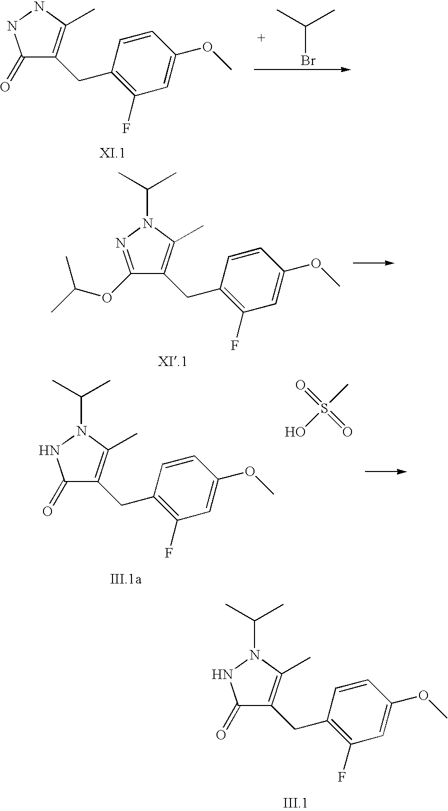

Step 1:

2-Bromopropane (144,5 ml; 1,52 mol) is added to a mixture of 1,2-dihydro 4-[(2-fluor-4-methoxyphenyl)methyl]-5-methyl-3H-pyrazol-3-one (90 g; 0,38 mol), powdered potassium hydroxide (88 g; 1,33 mol) and N-methylpyrrolidone (540 ml) at 5° C. within about 10 minutes. The reaction mixture is allowed to warm to about 20 to 25° C. and stirring is continued for 15 h. It is then poured onto a mixture of ice/water (1.5 L) and toluene (450 ml). By addition of aqueous concentrated hydrochloric acid (16.5 ml) a pH of approx. 3 is maintained. The phases are separated and the aqueous phase is extracted twice with toluene. The combined toluene phases are washed with water and brine, dried and evaporated under reduced pressure to yield an oil (orange). This is used in the next reaction without further purification.

Mass and ¹H-NMR-spectra are in accordance with the assigned structure of XI′.1.

Mass spectrum (ESI⁺): m/z=321 [M+H]⁺

Step 2:

A mixture of 1-(1-methylethyl)-3-(1-methylethoxy)-4-[(2-fluor-4-methoxyphenyl)-methyl]-5-methyl-pyrazole (132 g; 0.33 mol) and aqueous methanesulfonic acid (20%; 925 ml) is heated in a closed reactor to about 140° C. for about 5 hrs. It is then allowed to cool to about 20 to 25° C. The product is isolated by filtration, washed sequentially with water and isopropanol and dried at 500 to yield the product as pale crystals (yellow). Mass and ¹H-NMR-spectra are in accordance with the assigned structure of III.1a.

Mass spectrum (ESI⁺): m/z=279 [M+H]⁺

Step 3:

1,2-Dihydro-1-(1-methylethyl)-4-[(2-fluor-4-methoxyphenyl)methyl]-5-methyl-3H-pyrazol-3-one methanesulphonic acid salt (85 g; 0,23 mol) is treated with a mixture of dichloromethane (600 ml) and 4N aqueous sodium hydroxide (57 ml). The phases are separated. The organic phase is washed with brine, dried over sodiumsulphate, filtered and evaporated. The resulting residue is treated with boiling ethanol, filtered and dried at 50° C. Light beige crystals.

Mass and ¹H-NMR-spectra are in accordance with the assigned structure of the product III.1.

Mass spectrum (ESI⁺): m/z=279 [M+H]⁺

### EXAMPLE 59

### 1,2-Dihydro-1-(1-methylethyl)-4-[(2,3-difluoro-4-methoxyphenyl)methyl]-5-methyl-3H-pyrazol-3-one (III.2)

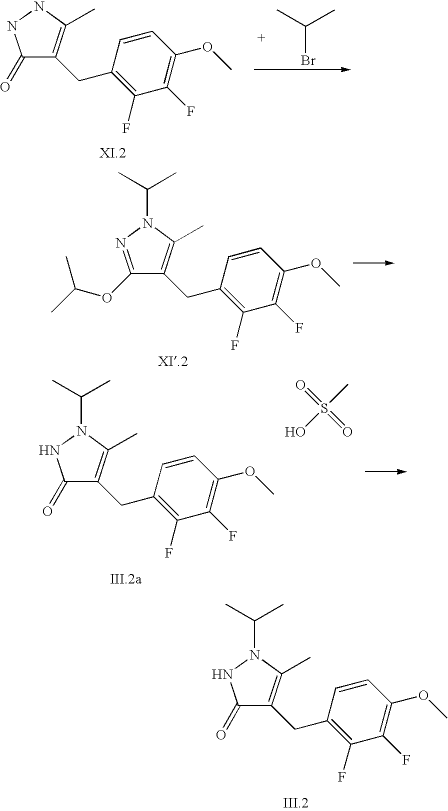

The compound of the formula III.2 may be obtained by employing the synthetic procedure as outlined in example 58 in an analogous manner.

### EXAMPLE 60

### 1,2-Dihydro-1-(1-methylethyl)-4-[(2,6-difluoro-4-methoxyphenyl)methyl]-5-methyl-3H-pyrazol-3-one (III.3)

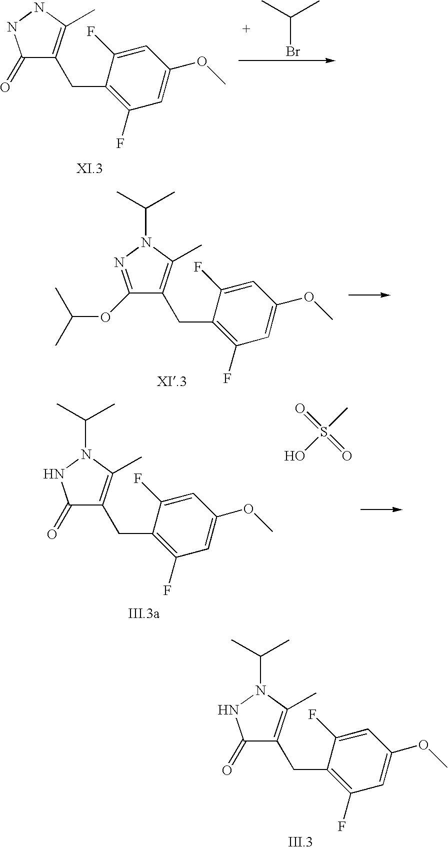

The compound of the formula III.3 may be obtained by employing the synthetic procedure as outlined in example 58 in an analogous manner.

### EXAMPLE 61

### 1,2-Dihydro-1-cyclobutyl-4-[(3-fluoro-4-methylphenyl)methyl]-5-methyl-3H-pyrazol-3-one (III.4)

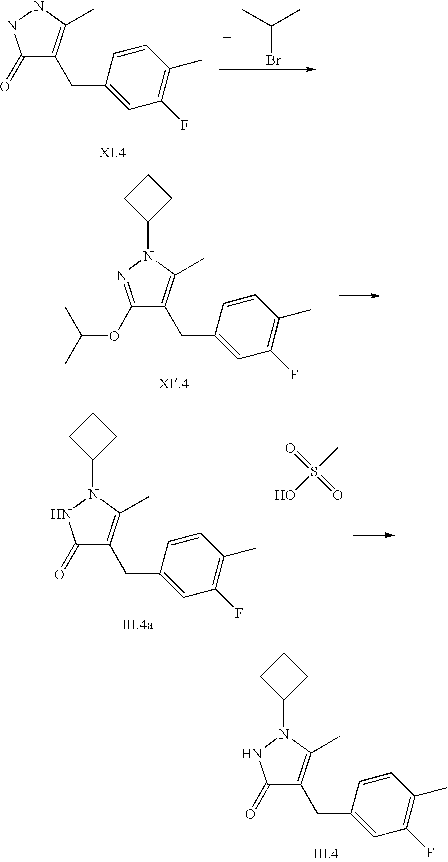

The compound of the formula III.4 may be obtained by employing the synthetic procedure as outlined in example 58 in an analogous manner.

### EXAMPLE 62

### 1,2-Dihydro-1-cyclobutyl-4-[(2-fluoro-4-methoxyphenyl)methyl]-5-methyl-3H-pyrazol-3-one (III.5)

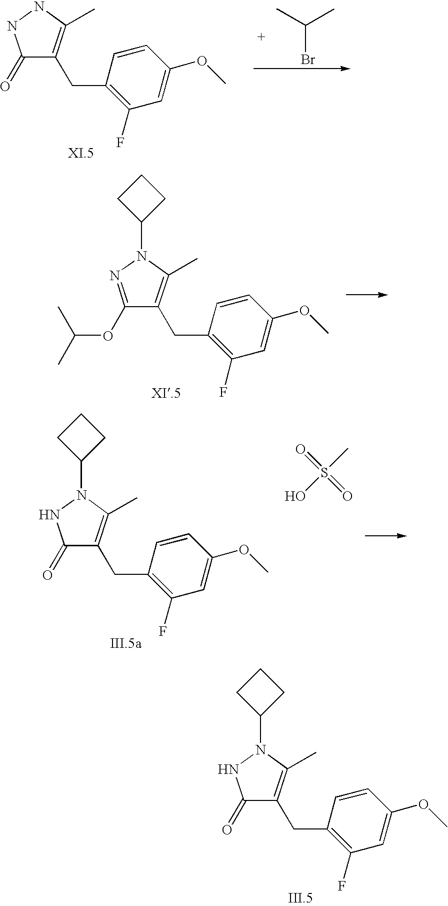

The compound of the formula III.5 may be obtained by employing the synthetic procedure as outlined in example 58 in an analogous manner.

### EXAMPLE 63

### 1,2-Dihydro-1-(1-methylethyl)-4-[(2,3-difluoro-4-methylphenyl)methyl]-5-methyl-3H-pyrazol-3-one (III.6)

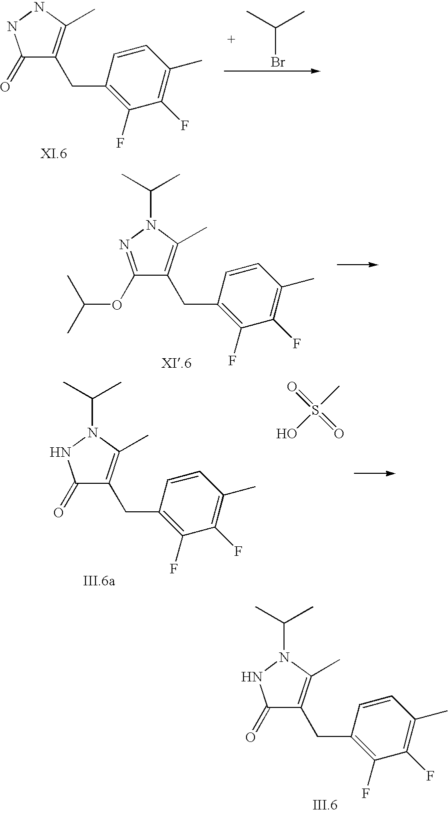

The compound of the formula III.6 may be obtained by employing the synthetic procedure as outlined in example 58 in an analogous manner.

### EXAMPLE 64

### 1,2-Dihydro-1-(1-methylethyl)-4-[(2-fluoro-4-methylphenyl)methyl]-5-methyl-3H-pyrazol-3-one (III.7)

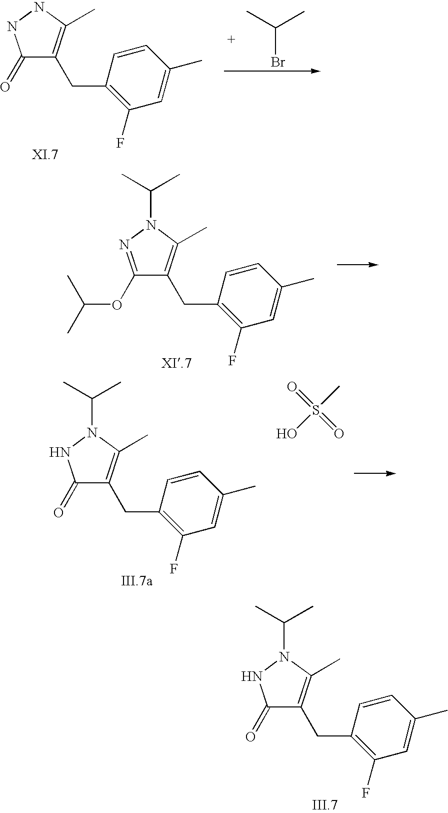

The compound of the formula III.7 may be obtained by employing the synthetic procedure as outlined in example 58 in an analogous manner.

### EXAMPLE 65

### 1,2-Dihydro-1-(1-methylethyl)-4-[(3-fluoro-4-ethoxyphenyl)methyl]-5-methyl-3H-pyrazol-3-one (III.8)

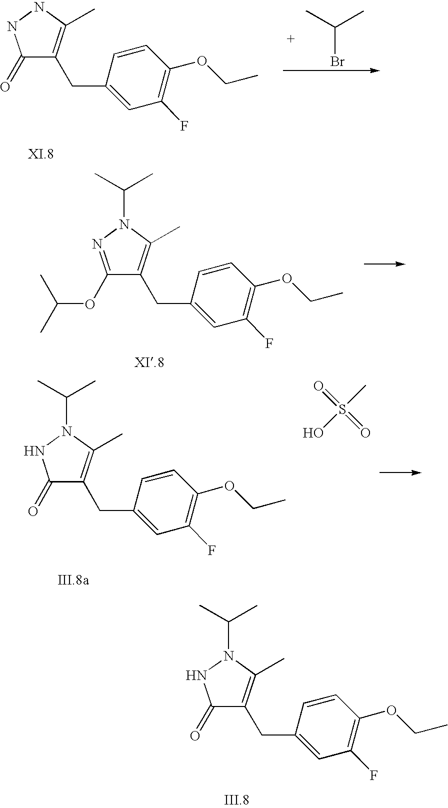

The compound of the formula III.8 may be obtained by employing the synthetic procedure as outlined in example 58 in an analogous manner.

### EXAMPLE 66

### 1,2-Dihydro-1-(1-methylethyl)-4-[(3-fluoro-4-(1-methylethoxy)phenyl)methyl]-5-methyl-3H-pyrazol-3-one (III.9)

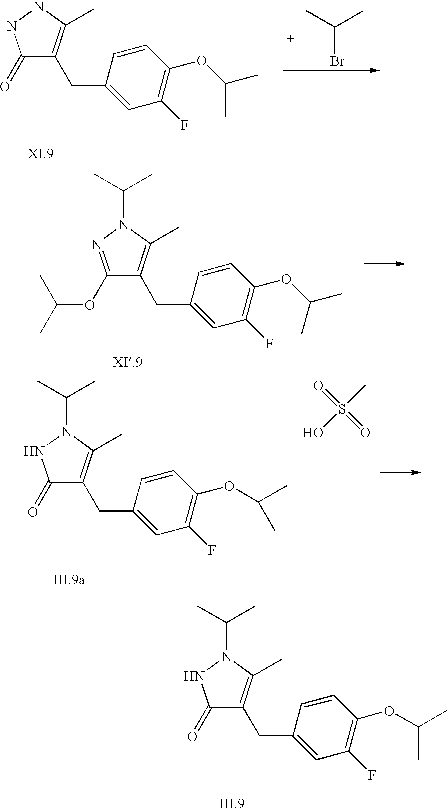

The compound of the formula III.9 may be obtained by employing the synthetic procedure as outlined in example 58 in an analogous manner.

### EXAMPLE 67

### 1,2-Dihydro-1-(1-methylethyl)-4-[(2-fluoro-4-(1-methylethoxy)phenyl)methyl]-5-methyl-3H-pyrazol-3-one (III.10)

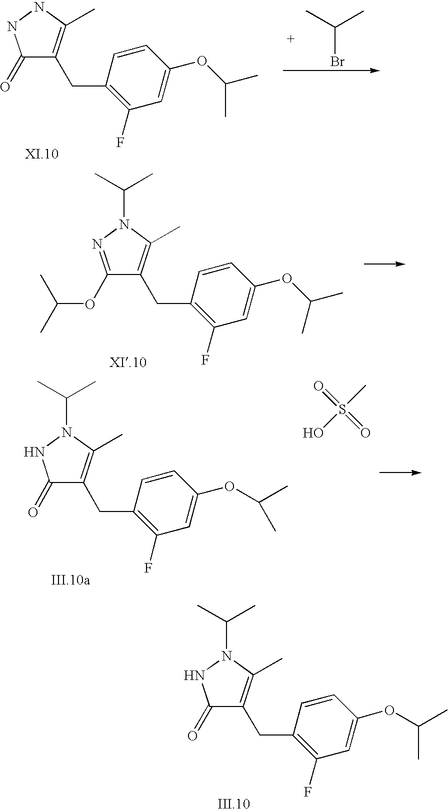

The compound of the formula III.10 may be obtained by employing the synthetic procedure as outlined in example 58 in an analogous manner.

### EXAMPLE 68

### 1,2-Dihydro-1-(1-methylethyl)-4-[(2-fluoro-4-ethoxyphenyl)methyl]-5-methyl-3H-pyrazol-3-one (III.11)

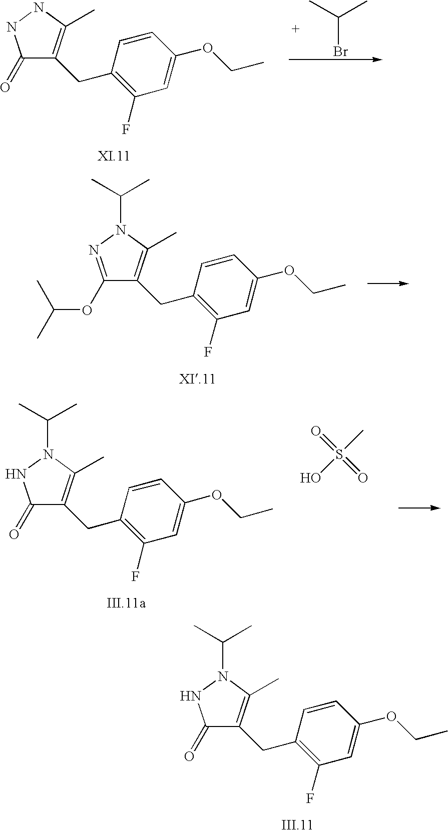

The compound of the formula III.11 may be obtained by employing the synthetic procedure as outlined in example 58 in an analogous manner.

1. Process for preparing compounds of the general formula (I), 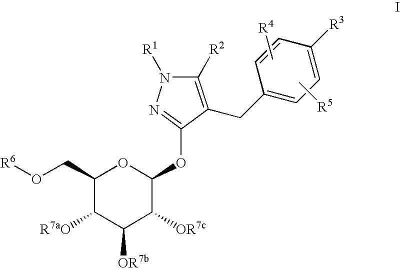
wherein
R¹ denotes C₁₋₄-alkyl, a C₁₋₄-alkyl group substituted with one or more fluorine atoms, or C₃₋₆-cycloalkyl; and
R² denotes C₁₋₄-alkyl, a C₁₋₄-alkyl group substituted with one or more fluorine atoms, or C₃₋₆-cycloalkyl; and
R³ denotes fluorine, chlorine, bromine, C₁₋₄-alkyl, C₃₋₆-cycloalkyl, C₁₋₄-alkoxy, or C₃₋₆-cycloalkyl-oxy; and
R⁴, R⁵ independently of one another denote hydrogen, fluorine, chlorine, bromine, C₁₋₄-alkyl, or C₁₋₄-alkoxy; and
R⁶ R^{7a},
R^{7b}, R^{7c} independently of one another have a meaning selected from the group hydrogen, (C₁₋₆-alkyl)carbonyl, phenylcarbonyl and phenyl-(C₁₋₃-alkyl)-carbonyl;
including the tautomers, stereoisomers, mixtures thereof and the salts thereof;
said method comprised of the steps of:
performing catalytic hydrolysis of a compound according to formula (IV) 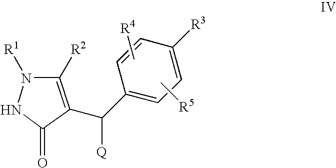
wherein R¹ to R⁵ are defined as hereinbefore, and
Q is Cl, Br, I, C₁₋₄-alkoxy, C₁₋₄-alkylthio, phenylthio, C₃₋₆-cycloalkyl-oxy, C₁₋₄-alkylcarbonyloxy, —NR^{a}R^{b}, wherein R^{a}, R^{b} independently of one another denote C₁₋₄-alkyl, or —NR^{a}R^{b} denotes pyrrolidinyl, piperidinyl, morpholinyl, piperazinyl or N—C₁₋₄-alkyl-piperazinyl;
on the aglycone of the formula (III) 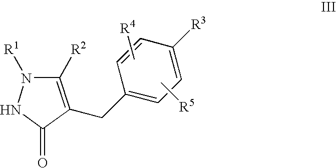
wherein R¹ to R⁵ are defined as hereinbefore.

2. Process according to claim 1 characterized in that the compound of the formula (IV) wherein Q denotes C₁₋₄-alkoxy, C₁₋₄-alkylthio, phenylthio, C₃₋₆-cycloalkyl-oxy or —NR^{a}R^{b}, wherein R^{a}, R^{b} independently of one another denote C₁₋₄-alkyl, or NR^{a}R^{b} denotes pyrrolidinyl, piperidinyl, morpholinyl, piperazinyl or N—C₁₋₄-alkyl-piperazinyl; is obtained by reacting a pyrazole derivative of the formula (VI) 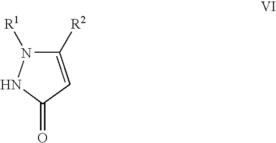
wherein R¹ and R² are defined as in claim 1,
with a benzaldehyde derivative of the formula (V) 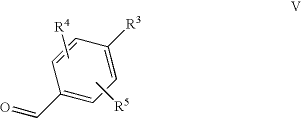
wherein R³, R⁴ and R⁵ are defined as in claim 1,
in the presence of either
a) a secondary amine H-Q, wherein Q denotes —NR^{a}R^{b}, wherein R^{a}, R^{b} independently of one another denote C₁₋₄-alkyl, or —NR^{a}R^{b} denotes pyrrolidinyl, piperidinyl, morpholinyl, piperazinyl or N—C₁₋₄-alkyl-piperazinyl; or
b) an alcohol or thiol H-Q, wherein Q denotes C₁₋₄-alkoxy, C₁₋₄-alkylthio, phenylthio or C₃₋₆-cycloalkyl-oxy, and a secondary amine.

3. Process according to claim 2 characterized in that the reaction is carried out in the presence of an alcohol H-Q, wherein Q is selected from among methoxy, ethoxy, n-propoxy and i-propoxy, and a cyclic secondary amine.

4. Process according to claim 2 characterized in that the pyrazole derivative of the formula (VI) is obtained by dehydrogenation of the pyrazole derivative of the formula (VII) 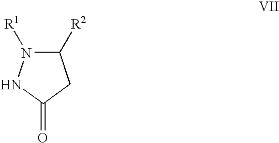
wherein R¹ and R² are defined as in claim 2.

5. Process according to claim 4 characterized in that the pyrazole derivative of the formula (VII) 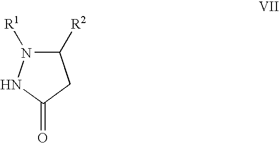
wherein R¹ denotes a group of the formula 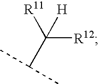
wherein R¹¹ denotes C₁₋₃-alkyl or C₁₋₃-alkyl substituted with one or more fluorine atoms; and R¹² denotes H, or in case R¹¹ denotes methyl, R¹² may also denote a methyl- or ethyl-group or a methyl- or ethyl-group substituted with one or more fluorine atomes; or R¹¹ and R¹² are linked and form together with the CH-group to which they are attached a C₃₋₆-cycloalkyl-group; R² is defined as in claim 4;
is obtained by reacting the pyrazole derivative of the formula (VII) 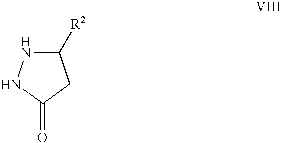
wherein R² is defined as hereinbefore;
with an aldehyde or ketone of the formula (IX) 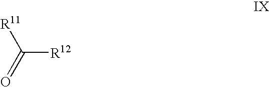
wherein R¹¹ and R¹² are defined as hereinbefore;
and subsequent or concomitant reduction.

6. Process according to claim 5 characterized in that the pyrazole derivative of the formula (VIII) is obtained by reacting an acrylic acid ester derivative of the formula (X) 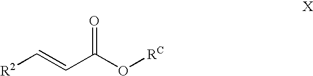
wherein R² is defined as in claim 5; and
R^{C} is methyl, ethyl, n-propyl or i-propyl;
with a hydrazine in a solvent or mixture of solvents.

7. Process according to claim 1 characterized in that the aglycone of the formula (III) according to claim 1 is reacted with a glucose derivative of the formula (II) 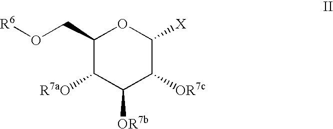
wherein
X denotes bromine or chlorine;
R⁶, R^{7a},
R^{7b} R⁷C independently of one another have a meaning selected from the group (C₁₋₆alkyl)carbonyl, phenylcarbonyl and phenyl-(C₁₋₃-alkyl)-carbonyl;
in a solvent or mixture of solvents to yield the compound of the general formula (I).

8. Process according to claim 7 characterized in that the product of the formula (I), wherein one or more substituents R⁶, R^{7a}, R^{7b}, R^{7c} are not hydrogen, is deprotected.

9. Process for preparing compounds of the formula (IH) 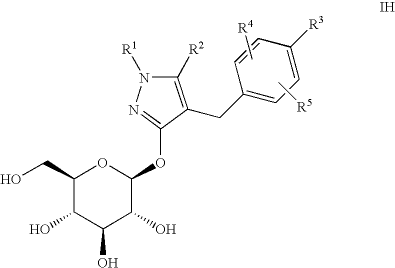
wherein R¹ to R⁵ are defined as in claim 1,
including the tautomers, stereoisomers, mixtures thereof and the salts thereof;
said method comprising the step of deprotecting the compound of the formula (I) 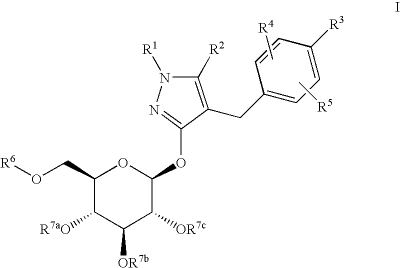
wherein R¹ to R⁵ are defined as hereinbefore and R⁶, R^{7a}, R^{7b} and R^{7c} are defined as in claim 1 but one or more of them not being hydrogen, by cleaving the substituents R⁶, R^{7a}, R^{7b} and R^{7c} not being hydrogen in a solvent or a mixture of solvents.

10. Process for preparing compounds of the formula (III) 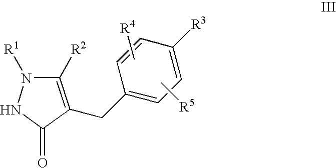
wherein
R¹ denotes C₁₋₄-alkyl, a C₁₋₄-alkyl group substituted with one or more fluorine atoms, or C₃₋₆-cycloalkyl; and
R² denotes C₁₋₄-alkyl, a C₁₋₄-alkyl group substituted with one or more fluorine atoms, or C₃₋₆-cycloalkyl; and
R³ denotes fluorine, chlorine, bromine, C₁₋₄-alkyl, C₃₋₆-cycloalkyl, C₁₋₄-alkoxy, or C₃₋₆-cycloalkyl-oxy; and
R⁴, R⁵ independently of one another denote hydrogen, fluorine, chlorine, bromine, C₁₋₄-alkyl, or C₁₋₄-alkoxy;
including the tautomers, stereoisomers, mixtures thereof and the salts thereof;
comprising the step of catalytically hydrogenating a pyrazole derivative of the formula (IV) 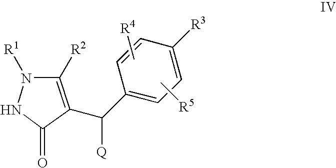
wherein R¹ to R⁵ are defined as hereinbefore; and
Q is Cl, Br, I, C₁₋₄-alkoxy, C₁₋₄-alkylthio, phenylthio, C₃₋₆-cycloalkyl-oxy, C₁₋₄-alkylcarbonyloxy, —NR^{a}R^{b}, wherein R^{a}, R^{b} independently of one another denote C₁₋₄-alkyl, or —NR^{a}R^{b} denotes pyrrolidinyl, piperidinyl, morpholinyl, piperazinyl or N—C₁₋₄-alkyl-piperazinyl,
in a solvent or a mixture of solvents.

11. Process for preparing compounds of the formula (IV) 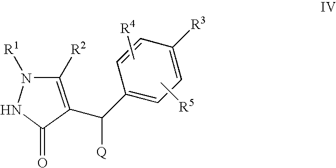
wherein
R¹ denotes C₁₋₄-alkyl, a C₁₋₄-alkyl group substituted with 1 to 3 fluorine atoms, or C₃₋₆-cycloalkyl; and
R² denotes C₁₋₄-alkyl, a C₁₋₄-alkyl group substituted with one or more fluorine atoms, or C₃₋₆-cycloalkyl; and
R³ denotes fluorine, chlorine, bromine, C₁₋₄-alkyl, C₃₋₆-cycloalkyl, C₁₋₄-alkoxy, or C₃₋₆-cycloalkyl-oxy; and
R⁴, R⁵ independently of one another denote hydrogen, fluorine, chlorine, bromine, C₁₋₄-alkyl, or C₁₋₄-alkoxy; and
Q is C₁₋₄-alkoxy, C₁₋₄-alkylthio, phenylthio, C₃₋₆-cycloalkyl-oxy, —NR^{a}R^{b}, wherein R^{a}, R^{b} independently of one another denote C₁₋₄-alkyl, or —NR^{a}R^{b} denotes pyrrolidinyl, piperidinyl, morpholinyl, piperazinyl or N—C₁₋₄-alkyl-piperazinyl;
including the tautomers, stereoisomers, mixtures thereof and the salts thereof;
comprising the step of reacting a pyrazole derivative of the formula (VI) 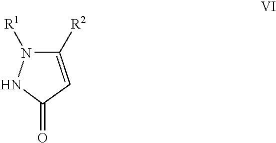
wherein R¹ and R² are defined as hereinbefore;
with a benzaldehyde derivative of the formula (V) 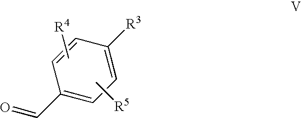
wherein R³, R⁴ and R⁵ are defined as hereinbefore,
in the presence of either
a) a secondary amine H-Q, wherein Q denotes —NR^{a}R^{b}, wherein R^{a}, R^{b} independently of one another denote C₁₋₄-alkyl or —NR^{a}R^{b} denotes pyrrolidinyl, piperidinyl, morpholinyl, piperazinyl or N—C₁₋₄-alkyl-piperazinyl; or
b) an alcohol or thiol H-Q, wherein Q denotes C₁₋₄-alkoxy, C₁₋₄-alkylthio, phenylthio or C₃₋₆-cycloalkyl-oxy, and a secondary amine.

12. Process for preparing compounds of the general formula (I), 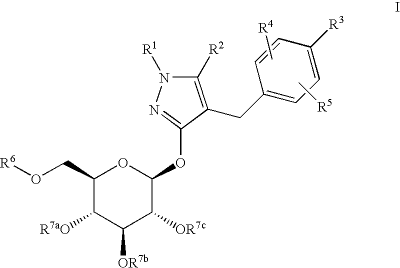
wherein R¹ to R⁵, R⁶, R⁷, R^{7b}, R^{7c} are defined as in claim 1;
including the tautomers, stereoisomers, mixtures thereof and the salts thereof;
characterized in that an aglycone of the formula (III) 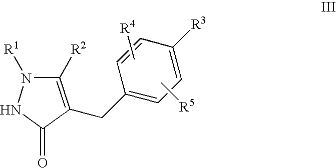
wherein R¹ to R⁵ are defined as hereinbefore;
is reacted with a glucose derivative of the formula (II) 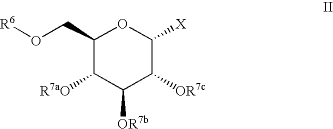
wherein
X denotes bromine or chlorine;
R⁶, R^{7a},
R^{7b} R^{7a}, independently of one another have a meaning selected from the group (C₁₋₄-alkyl)carbonyl, phenylcarbonyl and phenyl-(C₁₋₃-alkyl)-carbonyl;
in a solvent or mixture of solvents.

13. Process according to claim 12 characterized in that the product of the formula (I), wherein one or more of the substituents R⁶, R^{7a}, R^{7b}, R^{7c} are not hydrogen.

14. Process according to claim 12 characterized in that the aglycone of the formula (III) is obtained by a process starting from a pyrazole derivative of the formula (IV)

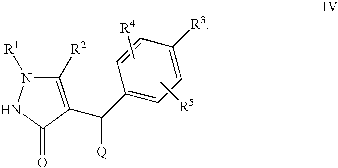

15. Process according to claim 14 characterized in that the pyrazole derivative of the formula (IV) is obtained by a process according to claim 11.

16. Process according to claim 12 characterized in that the aglycone of the formula (III) is obtained by reacting a pyrazole derivative of the formula (XI) 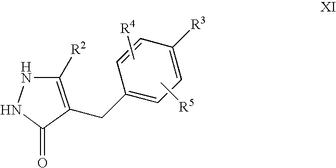
wherein R² to R⁵ are defined as in claim 12;
with an alkylating agent R¹—X′ wherein R¹ is defined as in claim 12 and X′ denotes chlorine, bromine, iodine or C₁₋₃-alkyl-SO₂—O—, in the presence of a base in a solvent or mixture of solvents yielding an intermediate of the formula (XI′) 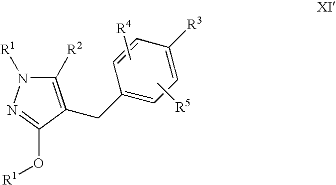
wherein R¹ to R⁵ are defined as hereinbefore;
and subsequent cleaving the R¹—O-group at the 3-position of the pyrazole ring, in particular in the presence of an acid, yielding the aglycone of the formula (III).

17. Process according to claim 16 characterized in that the pyrazole derivative of the formula (XI) is obtained by
(i) reacting a benzaldehyde derivative of the formula (V) 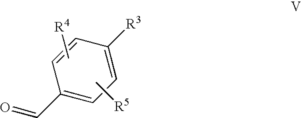
wherein R³, R⁴ and R⁵ are defined as in claim 16,
with a β-ketoester derivative of the formula (XII) 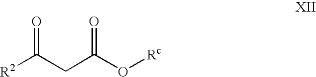
wherein R² is defined as in claim 16; and
R^{C} is methyl, ethyl, n-propyl or i-propyl;
in the presence of an acid and a secondary amine and subsequent or concomitant catalytic hydrogenation; and
(ii) reacting the product of step (i) with hydrazine in a solvent or mixture of solvents.

18. Process for preparing a pyrazole derivative of the formula (III) 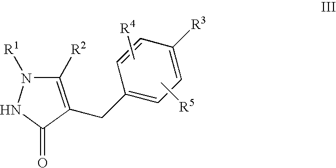
wherein R¹ to R⁵ are defined as in claim 1;
including the tautomers, stereoisomers, mixtures thereof and the salts thereof;
comprising reacting a pyrazole derivative of the formula (XI) 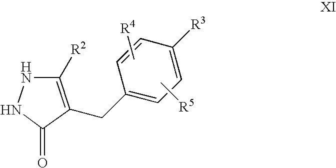
wherein R² to R⁵ are defined as before;
with an alkylating agent R¹—X′ wherein R¹ is defined in claim 1 and X′ denotes chlorine, bromine, iodine or C₁₋₃-alkyl-SO₂—O—, in the presence of a base in a solvent or mixture of solvents yielding an intermediate of the formula (XI′) 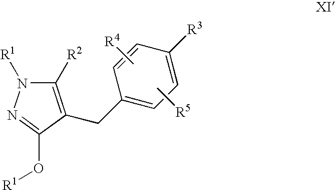
wherein R¹ to R⁵ are defined as hereinbefore;
and subsequent cleaving the R¹—O-group at the 3-position of the pyrazole ring, in particular in the presence of an acid yielding the aglycone of the formula (III).

19. Process for preparing a pyrazole derivative of the formula (XI) 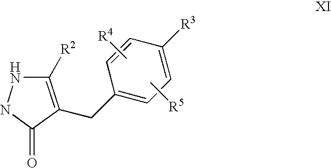
wherein R² to R⁵ are defined as in claim 1;
including the tautomers, stereoisomers, mixtures thereof and the salts thereof;
comprising the steps:
(i) reacting a benzaldehyde derivative of the formula (V) 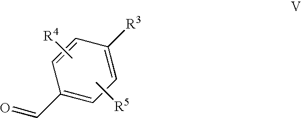
wherein R³, R⁴ and R⁵ are defined as hereinbefore,
with a β-ketoester derivative of the formula (XII) 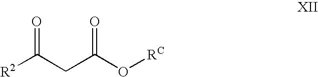
wherein R² is defined as hereinbefore; and
R^{C} is methyl, ethyl, n-propyl or i-propyl;
in the presence of an acid and a secondary amine and subsequent or concomitant catalytic hydrogenation; and
(ii) reacting the product of step (i) with hydrazine in a solvent or mixture of solvents.

20. Process for preparing compounds of the general formula (I) as defined in claim 1 comprising the step of reacting a pyrazole derivative of the formula (VI) 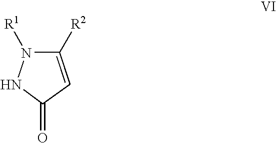
wherein R¹ and R² are defined as hereinbefore;
with a benzaldehyde derivative of the formula (V) 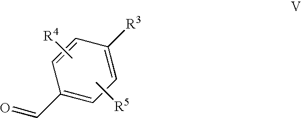
wherein R³, R⁴ and R⁵ are defined as hereinbefore,
in the presence of either
a) a secondary amine H-Q, wherein Q denotes —NR^{a}R^{b}, wherein R^{a}, R^{b} independently of one another denote C₁₋₄-alkyl or —NR^{a}R^{b} denotes pyrrolidinyl, piperidinyl, morpholinyl, piperazinyl or N—C₁₋₄-alkyl-piperazinyl; or
b) an alcohol or thiol H-Q, wherein Q denotes C₁₋₄-alkoxy, C₁₋₄-alkylthio, phenylthio or C₃₋₆-cycloalkyl-oxy, and a secondary amine.

21. Process for preparing compounds of the general formula (I) as defined in claim 1 comprising preparing a pyrazole derivative of the formula (III) 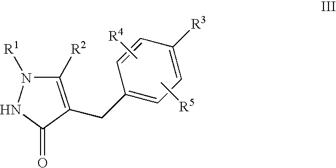
wherein R¹ to R⁵ are defined as in claim 1;
including the tautomers, stereoisomers, mixtures thereof and the salts thereof;
comprising reacting a pyrazole derivative of the formula (XI) 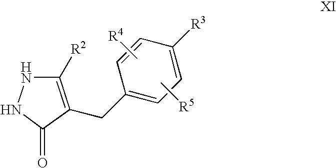
wherein R² to R⁵ are defined as before;
with an alkylating agent R¹—X′ wherein R¹ is defined in claim 1 and X′ denotes chlorine, bromine, iodine or C₁₋₃-alkyl-SO₂—O—, in the presence of a base in a solvent or mixture of solvents yielding an intermediate of the formula (XI′) 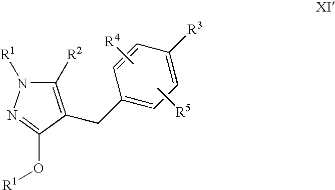
wherein R¹ to R⁵ are defined as hereinbefore;
and subsequent cleaving the R¹—O-group at the 3-position of the pyrazole ring, in particular in the presence of an acid yielding the aglycone of the formula (III).

22. Process for preparing compounds of the general formula (I) as defined in claim 1 comprising preparing a pyrazole derivative of the formula (XI) 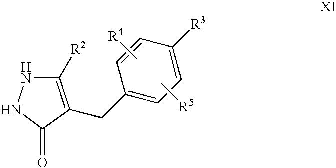
wherein R² to R⁵ are defined as in claim 1;
including the tautomers, stereoisomers, mixtures thereof and the salts thereof;
comprising the steps:
(i) reacting a benzaldehyde derivative of the formula (V) 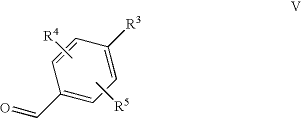
wherein R³, R⁴ and R⁵ are defined as hereinbefore,
with a β-ketoester derivative of the formula (XII) 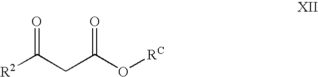
wherein R² is defined as hereinbefore; and
R^{C} is methyl, ethyl, n-propyl or i-propyl;
in the presence of an acid and a secondary amine and subsequent or concomitant catalytic hydrogenation; and
(ii) reacting the product of step (i) with hydrazine in a solvent or mixture of solvents.

23. Process according to claim 1 characterized in that the substituents where applicable are defined as follows:
R¹ denotes methyl, ethyl, n-propyl, i-propyl, cyclobutyl or cyclopentyl; and
R² denotes methyl, ethyl, n-propyl or i-propyl; and
R³ denotes fluorine, chlorine, methyl, methoxy, ethoxy, n-propoxy or i-propoxy; and
R⁴ denotes fluorine, chlorine, methyl, methoxy, ethoxy, n-propoxy or i-propoxy; and
R⁵ denotes hydrogen, fluorine, chlorine, methyl or methoxy.

24. Process according to claim 21 characterized in that the substituents where applicable are defined as follows:
R¹ denotes i-propyl or cyclobutyl; and
R² denotes methyl; and
R³ denotes methyl, methoxy, ethoxy or i-propoxy; and
R⁴ denotes fluorine; and
R⁵ denotes hydrogen or fluorine.

25. Process according to claim 1 characterized in that the substituents where applicable are defined as follows:
in the formula (I) the substituents R⁶, R⁷, R^{7b}, R^{7c} denote hydrogen; and
in the formula (II) the substituents R⁶, R⁷, R^{7b}, R^{7c} denote (C₁₋₄-alkyl)carbonyl, in particular methylcarbonyl; and
in the formula (II′) the substituents R⁶, R⁷, R^{7b}, R^{7c}, R^{7d} denote (C₁₋₄-alkyl)carbonyl, in particular methylcarbonyl.

26. A compound of the formula (IV) 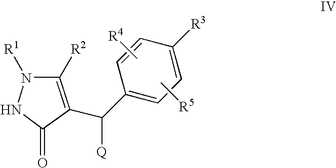
wherein R¹ to R⁵ are defined as in claim 1 and
Q is Cl, Br, I, C₁₋₄-alkoxy, C₁₋₄-alkylthio, phenylthio, C₃₋₆-cycloalkyl-oxy, C₁₋₄-alkylcarbonyloxy, —NR^{a}R^{b}, wherein R^{a}, R^{b} independently of one another denote C₁₋₄-alkyl, or —NR^{a}R^{b} denotes pyrrolidinyl, piperidinyl, morpholinyl, piperazinyl or N—C₁₋₄-alkyl-piperazinyl,
including the tautomers, the stereoisomers, the mixtures thereof and the salts thereof.

27. A compound of the formula (III) 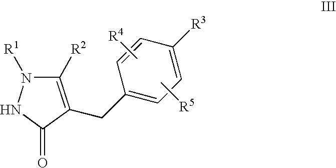
wherein R¹ to R⁵ are defined as in claim 1 including the tautomers, the stereoisomers, the mixtures thereof and the salts thereof.

28. A compound of the formula (VI) 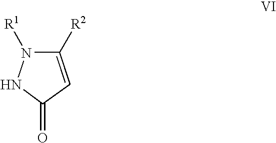
wherein R¹ and R² are defined as in claim 1 including the tautomers, the mixtures thereof and the salts thereof.
